(19) United States
(12) Patent Application Publication
(10) Pub. No.: US 2017/0238550 A1
(43) Pub. Date: Aug. 24, 2017

(54) Aqueous Alcoholic Microbicidal Compositions Comprising Copper Ions

(71) Applicant: Reckitt Benckiser LLC, Parsippany, NJ (US)

(72) Inventors: Mohammad Khalid IJAZ, New Milford, NJ (US); Joseph RUBINO, Parsippany, NJ (US); Yun-Peng ZHU, Fairlawn, NJ (US)

(21) Appl. No.: 15/591,787

(22) Filed: May 10, 2017

Related U.S. Application Data
(62) Division of application No. 14/122,434, filed on Jan. 28, 2014.
(60) Provisional application No. 61/492,000, filed on Jun. 1, 2011.

(51) Int. Cl. A01N 59/20; A61L 2/18; A61L 9/14; A01N 31/02

(52) U.S. Cl. CPC A01N 59/20; A01N 31/02; A61L 2/18; A61L 9/145

(57) ABSTRACT

Compositions which impart a microbicidal benefit to treated surfaces which compositions comprise (or in certain preferred embodiments may consist essentially of, or may consist of): a copper source material which releases copper ions into the treatment composition, preferably a source of Cu(I) and/or Cu(II) ions; preferably at least from about 20% wt. of at least one alcohol, which is preferably a lower alkyl monohydric alcohol) and water, optionally one or more further surfactants, further optionally one or more constituents which impart one or more advantageous technical or aesthetic benefits to the compositions, including one or more detersive surfactants;, and water, wherein the composition has a pH of at least 4, preferably at least 5, wherein the surface treatment compositions, exhibit a microbicidal or germicidal or antimicrobial effect on treated inanimate surfaces or when used to treat an airspace, e.g. ambient air,. characterized in exhibiting a microbicidal benefit when tested against one or more challenge microorganisms, according to one or more of the following standardized test protocols: ASTM E1052 Standard Test Method for Efficacy of Antimicrobial Agents against Viruses in Suspension, or ASTM E1053 Standard Test Method to Assess Virucidal Activity of Chemicals Intended for Disinfection of Inanimate, Nonporous Environmental Surfaces, or European Standard Surface Test, EN13697, or AOAC Germicidal Spray Products as Disinfectant Test Method, AOAC Index, 17^{th} Ed. (2000, preferably against poliovirus type 1 (Sabin) (“PV1”)

The present invention relates to compositions which comprise copper ions which compositions exhibit a microbicidal benefit when applied to inanimate surfaces or when used to treat the air. The aqueous alcoholic compositions provide a surprisingly high degree of microbicidal activity against various undesirable microorganisms (sometimes referred to as ‘pathogens’) including various bacteria, mycobacteria, viruses, and fungi.

While ethanol and other monohydric alcohols are known to the art as having a beneficial microbicidal benefit, at the same time it is a volatile organic compound (“VOC”) and there is a substantial interest in regulating the use of ethanol (as well as other volatile organic compounds) in products wherein the ethanol or other VOC is exposed to the environment. Such regulatory interests are however completely contrary to the technical benefits provided by ethanol and other monohydric alcohols, and in particular ethanol, as a microbicidal agent, as increased levels of ethanol in a composition have long been known to find increased microbicidal benefits against undesirable microorganisms.

The technical art has proposed several compositions which are lauded to provide some degree of microbicidal/germicidal/antimicrobial efficacy, at the same time to comprise reduced amounts of ethanol and other monohydric alcohols while still providing an appreciable microbicidal benefit. However, these compositions are not wholly successful in providing a microbicidal/germicidal/antimicrobial benefit against a broad range of undesirable microorganisms, and in particular in providing effective microbicidal benefit against particularly difficult to eradicate microorganisms including non-enveloped viruses, and in particular polioviruses [Poliovirus (e.g., poliovirus type 1 (Sabin Type 1).] As is recognized in the art, demonstrated eradication of poliovirus is highly advantageous as such compositions would not only be effective in controlling this dangerous microorganism but at the same time such a high level of efficacy would also be recognized as having a high degree of relatively easier to eradicate microorganisms including but not limited to bacteria, mycobacteria, other non-enveloped and enveloped virus strains including and in many cases, fungi.

The prior art discloses various compositions which are cited to provide a microbicidal effect. For example, in U.S. Pat. No. 5,180,749 are described largely aqueous compositions comprising about 65-88% wt. water, and which include as further essential constituents both about 10-30% wt. ethanol with about 2-5% wt. benzyl alcohol, but the use of water soluble metal salts is not disclosed nor is the pH of the compositions disclosed. The compositions were tested against Staphylococcus aureus, Salmonella choleraesuis, Pseudomonas aeruginosa, Rhinovirus Type 39, herpes simplex 1, herpes simplex 2, adenovirus type 2, respiratory syncytial, influenza A2, influenza B, human rotavirus, Mycobacterium tuberculosis var. bovis, as well as fungi of types Aspergillus niger and Trichopython mentgrophytes. In that patent, the data from Table B as contrasted to the data from Table A, the necessary inclusion of benzyl alcohol in conjunction with ethanol in order to achieve increased microbicidal efficacy is shown. The poor microbicidal efficacy of compositions comprising 30% wt. ethanol and water and where benzyl alcohol is absent is demonstrated on Table B. In U.S. Pat. No. 3,992,146 are disclosed germicidal and antifungal compositions which are based on aqueous solutions of a copper compound and a surfactant. The surfactants disclosed are primarily anionic surfactants based on sulfate or sulfonated organic compounds. The use of ethanol or of specific pH ranges are not clearly disclosed or demonstrated.

U.S. Pat. No. 5,728,404 discloses certain virucidal disinfectant compositions which are described as including one or more C₁-C₄ aliphatic alcohols, 0.1-1% wt. of a hydrolized metal ion, and water. Compositions comprising ethyl alcohol and isopropyl alcohol and ratios of 8:1 to 1:1 are noted to be particularly effective and preferred. While the document alleges that the amount of the aliphatic alcohol may be in the range of 40% -90% wt., such is not demonstrated as in the four examples provided the amount of the aliphatic alcohols are respectively 80% wt., 70% wt., 80% wt. and 80% wt. Furthermore, when formed as described in that document, the composition according to Example 1 of this patent document exhibited a pH of 5.48, the composition of Example 2 exhibited a pH of 5.63, and the composition of Example 3 exhibited a pH of 5.63, which indicates that the foregoing compositions consistently demonstrated an acidic pH.

U.S. Pat. No. 6,034,043 and U.S. Pat. No. 6,017,861 disclose liquid skin cleaning compositions comprising (1) a so-called mild surfactant system, of which at least 10% wt. of which (preferably at least 25% wt. of which,), is an anionic surfactant, (2) 0.1-10% wt. of a polyvalent cation or cations selected from zinc, copper, tin, aluminum, cobalt, nickel, chromium, titanium, and/or manganese and mixtures thereof, and (3) 1-99% wt. water wherein the cations provide antimicrobial activity. These patents suggest that microbicidal activity of the liquid skin cleaning compositions was due to the combination of the mild surfactant system with the polyvalent cation or cations which in combination, provided a microbicidal benefit whereas the polyvalent cation or cations themselves did not provide a microbiocidal benefit. Further, none of the demonstrated compositions include lower alkyl monohydric alcohols.

US 2004/0213750 discloses aqueous alcoholic compositions which comprise 40% wt.-70% wt. of a lower alkanol, optionally a quaternary ammonium cationic compound which itself provides germicidal properties, water and a pH adjusting agent to provide a final pH of between 7 and 13. The compositions are shown to be effective against various microorganisms including gram-positive and gram-negative types of pathogenic bacteria, as well as Poliovirus (Type 1) at a 10 minute contact time. The reference however makes no mention of the use of copper ions in the compositions.

US 2007/0184013 discloses compositions which are cited to be effective against non-enveloped virus particles. The compositions comprise a C₁-C₆ alcohol and an efficacy-enhancing amount of one or more of: cationic oligomers and polymers, proton donors, chaotropic agents, and mixtures thereof with the proviso that when the compositions include a proton donor that a cationic oligomer or polymer is also present. The cationic oligomers and polymers disclosed are defined to include cationic polyalkylene imines, cationic ethoxy polyalkylene imines, cationic poly[N-[3-(dialkylammonio)alkyl]N′[3-(alkyleneoxyalkylene dialkylammonio)alkyl]urea dichloride], vinyl caprolactam/VP/dialkylaminoalkyl alkylate copolymers and polyquaternium copolymers. The example compositions disclosed in the reference demonstrate compositions having 62% wt. and even greater amounts of the C1-C6 alcohol as being present.

US 2008/0045491 discloses certain surface sanitizer compositions which are described as comprising 50%-90% wt. of a water miscible alcohol component, and an acid component to maintain the pH below about 5, a multivalent cation and the balance being water. The multivalent cation is described as including polymers having at least two positive charges such as polyamines, chitosan, polylysine, metal ions in metal compounds.

The treatment of biofilms by compositions which include certain heavy metals are known from US 2008/0118573. The treatment steps require that the biofilms be contacted with the said compositions be contacted for 4 hours or more. The biofilms are defined to be conglomerates of microbial organisms embedded in highly hydrated matricies of exopolymers, typically polysaccharides, and other macromolecules.

US 2009/0226494 discloses certain antibacterial formulations which comprise a water-soluble copper compound, a water-soluble ammonium agent, and a water-soluble acid wherein the composition necessarily has an acidic pH.

US 2010/0233098 discloses methods and compositions for disinfecting hard surfaces which are aqueous compositions which comprise 40% wt.-70% wt. of an alcohol constituent selected from the group consisting of methanol, ethanol, n-propanol, isopropanol, n-butanol, benzyl alcohol, and mixtures thereof and a pH in the range of from about 7.0-14.0. The compositions may include further optional constituents, including ancillary antimicrobial agents, and surfactants, but the use of water soluble metal salts is not disclosed. US 2008/0045491 disclosed certain surface sanitizer compositions which are recited to include 50-90% wt. of an alcohol component, 10-50% wt. of water, an acid component to maintain the pH of the composition between 2-5, and 0.05-5% wt. of a multivalent cation constituent. The multivalent cation constituent may be one of a selected list of polymers, a metal ion or, a metal compound. The compositions may further optionally include one or more further constituents, including oxidative agents, plant derived alkenes or essential oils, emollients, humectants, lubricants and one or more antimicrobial compounds, e.g., quaternary ammonium compounds. A single example of US 2008/0045491 demonstrates that a composition having 78% wt. ethanol exhibits efficacy against Candida albicans, Aspergillus niger, Escherichia coli, Pseudomonas aeruginosa, Staphylococcus aureus and adenovirus type 5. Further examples disclosed in US 2008/0045491 are not disclosed to have been tested against any microorganisms.

Notwithstanding these various known art compositions, there is still an urgent need in the art to produce treatment compositions, particularly those adapted for the control or eradication of undesired microorganisms where such treatment compositions comprise reduced amounts of VOC, and in particular aliphatic alcohols which provide a microbicidal effect such as ethanol, yet which compositions are highly effective against particularly difficult to eradicate undesired microorganisms, especially poliovirus, particularly where the treatment compositions are applied to an inanimate surface or is used to treat an airspace.

In a broad aspect, the compositions of the present invention are generally directed to liquid inanimate surface treatment compositions which impart an antimicrobial or microbicidal benefit to treated surfaces which compositions comprise (or in certain preferred embodiments may consist essentially of, or may consist of): water, a copper source material which releases copper ions into the treatment composition, and at least one alcohol which independently of other constituents present exhibits a microbicidal effect. In certain embodiments, the compositions further additionally comprise at least one quaternary ammonium compound which provides a microbicidal benefit. In still further embodiments, the compositions further additionally comprise at least one detersive surfactant, and wherein the compositions are at a pH such that they exhibit a microbicidal effect on treated surfaces, or when used to treat an airspace, e.g., ambient air. These aforesaid compositions, further optionally include one or more further constituents which impart one or more advantageous technical or aesthetic benefits to the compositions. The compositions are at a pH such that the liquid inanimate surface treatment compositions, exhibit a microbiocidal or antipathogenic effect on treated surfaces or when used to treat an airspace, e.g. ambient air. Preferably these treatment compositions are characterized in exhibiting a microbicidal benefit when tested against one or more challenge organisms according to one or more of the following standardized test protocols: ASTM E1052 Standard Test Method for Efficacy of Antimicrobial Agents against Viruses in Suspension, or ASTM E1053 Standard Test Method to Assess Virucidal Activity of Chemicals Intended for Disinfection of Inanimate, Nonporous Environmental Surfaces, or European Standard Surface Test, EN13697, or AOAC Germicidal Spray Products as Disinfectant Test Method, AOAC Index, 17^{th} Ed. (2000) against one or more challenge microorganisms. Preferably the compositions are liquid, or sprayable liquid compositions (e.g. pumpable but are not aerosol compositions) and exhibit a viscosity of not more than about 100 cPs, preferably 50 cPs, more preferably 10 cPs at 20° C. when tested according to conventional quantitative methods (e.g., Brookfield Viscometer) and are pourable, readily flowable liquids. Such may be provided in any other apparatus or device wherein the liquid composition may be poured or sprayed onto a surface or into the air. The inventive compositions provide a high degree of microbicidal activity against various undesirable microorganisms (sometimes referred to as ‘pathogens’) including various bacteria, mycobacteria, viruses, and fungi.

Within this broad aspect, the present inventors have surprisingly observed that there may be formed compositions which exhibit a synergistic improvement in microbiocidal effect when there are added to aqueous alcoholic liquid compositions at specific pH ranges, especially preferably at alkaline pH ranges, small but effective amounts of a material which provides a copper ion to the aqueous alcoholic liquid compositions and (optionally but in most cases), especially wherein at least one further surfactant is also present. Such an effect is surprising, and also particularly technically advantageous, as improved microbiocidal efficacy has been observed against particularly difficult to control (or eradicate) microorganisms and in particular the poliovirus, while at the same time achieving these effects in aqueous alcoholic liquid compositions having a reduced VOC content. As is known to the art, non-enveloped viruses including poliovirus is particularly difficult to control or eradicate, and demonstrated microbiocidal efficacy against poliovirus is expected to be indicative of microbicidal efficacy against other non-enveloped viruses and microorganisms which are less difficult to control or eradicate.

The compositions of the present invention are directed to inanimate surface treatment compositions which impart a microbicidal benefit to treated surfaces which compositions comprise (or in certain preferred embodiments may consist essentially of, or may consist of): a copper source material which releases copper ions into the treatment composition, at least one alcohol which independently of other constituents present exhibits a microbicidal effect, and a liquid carrier which may be water or a mixture of water and an alcohol. The surface treatment compositions may further optionally comprise one or more additional constituents which impart one or more advantageous technical or aesthetic benefits to the compositions, including one or more detersive surfactants, wherein the compositions are at a pH such that the surface treatment compositions exhibit a microbicidal effect on treated surfaces. Preferably the surface treatment compositions are characterized in exhibiting a microbicidal benefit when tested according to one or more of the following standardized test protocols: ASTM E1052-96(2002) Standard Test Method for Efficacy of Antimicrobial Agents against Viruses in Suspension, or ASTM E1053-11 Standard Test Method to Assess Virucidal Activity of Chemicals Intended for Disinfection of Inanimate, Nonporous Environmental Surfaces, or European Standard Surface Test, EN13697, or AOAC Germicidal Spray Products as Disinfectant Test Method, AOAC Index, 17^{th} Ed. (2000) against one or more challenge microorganisms.

In a first aspect the present invention provides liquid, inanimate surface treatment compositions which impart a microbicidal benefit to treated surfaces which compositions comprise (or in certain preferred embodiments may consist essentially of, or may consist of):

a copper source material which releases copper ions into the treatment composition, preferably a source of Cu(I) and/or Cu(II) ions;

at least one lower alkyl aliphatic monohydric alcohol;

water;

optionally, one or more further constituents which impart one or more advantageous technical or aesthetic benefits to the compositions, including one or more detersive surfactants;

wherein the composition has a pH of at least 5,

wherein the surface treatment compositions are characterized in exhibiting a microbicidal benefit when tested against one or more challenge microorganisms according to one or more of the following standardized test protocols: ASTM E1052 Standard Test Method for Efficacy of Antimicrobial Agents against Viruses in Suspension, or ASTM E1053 Standard Test Method to Assess Virucidal Activity of Chemicals Intended for Disinfection of Inanimate, Nonporous Environmental Surfaces, or European Standard Surface Test, EN1369, or AOAC Germicidal Spray Products as Disinfectant Test Method, AOAC Index, 17^{th} Ed. (2000), especially preferably against poliovirus type 1 (Sabin) (“PV1”).

In a second aspect the present invention provides liquid, inanimate surface treatment compositions which impart a microbicidal benefit to such treated surfaces which compositions comprise (or in certain preferred embodiments may consist essentially of, or may consist of):

a copper source material which releases copper ions into the treatment composition, preferably a source of Cu(I) and/or Cu(II) ions;

in excess of 0% wt., and up to but excluding 20% wt. of at least one lower alkyl aliphatic monohydric alcohol;

optionally but preferably also at least one quaternary ammonium surfactant compound which provides a microbicidal benefit;

water;

optionally, one or more further constituents which impart one or more advantageous technical or aesthetic benefits to the compositions, including one or more detersive surfactants;

wherein the composition has a pH of at least 5,

wherein the surface treatment compositions are characterized in exhibiting a microbicidal benefit when tested against one or more challenge microorganisms according to one or more of the following standardized test protocols: ASTM E1052 Standard Test Method for Efficacy of Antimicrobial Agents against Viruses in Suspension, or ASTM E1053 Standard Test Method to Assess Virucidal Activity of Chemicals Intended for Disinfection of Inanimate, Nonporous Environmental Surfaces, or European Standard Surface Test, EN1369, or AOAC Germicidal Spray Products as Disinfectant Test Method, AOAC Index, 17^{th} Ed. (2000), especially preferably against poliovirus type 1 (Sabin) (“PV1”).

In a third aspect the present invention provides liquid, inanimate surface treatment compositions which impart a microbicidal benefit to such treated surfaces which compositions comprise (or in certain preferred embodiments may consist essentially of, or may consist of):

a copper source material which releases copper ions into the treatment composition, preferably a source of Cu(I) and/or Cu(II) ions;

at least one lower alkyl aliphatic monohydric alcohol;

at least one further detersive surfactant, other than the least one quaternary ammonium compound, which provides a microbicidal benefit, as compared to where such at least one such further detersive surfactant is absent, which is preferably at least one nonionic surfactant;

water;

optionally, one or more further constituents which impart one or more advantageous technical or aesthetic benefits to the compositions, including one or more detersive surfactants;

wherein the composition has a pH of at least 5,

wherein the surface treatment compositions are characterized in exhibiting a microbicidal benefit when tested according to one or more of the following standardized test protocols: ASTM E1052-96(2002) Standard Test Method for Efficacy of Antimicrobial Agents against Viruses in Suspension, or ASTM E1053-11 Standard Test Method to Assess Virucidal Activity of Chemicals Intended for Disinfection of Inanimate, Nonporous Environmental Surfaces, or European Standard Surface Test, EN1369, or AOAC Germicidal Spray Products as Disinfectant Test Method, AOAC Index, 17^{th} Ed. (2000) against one or more challenge microorganisms, especially preferably against Poliovirus type 1 (Sabin) (“PV1”).

In a fourth aspect the present invention provides liquid, inanimate surface treatment compositions which impart a microbicidal benefit to such treated surfaces which compositions comprise (or in certain preferred embodiments may consist essentially of, or may consist of):

a copper source material which releases copper ions into the treatment composition, preferably a source of Cu(I) and/or Cu(II) ions;

at least one lower alkyl aliphatic monohydric alcohol;

at least one quaternary ammonium surfactant compound which provides a microbicidal benefit;

at least one further detersive surfactant, other than the least one quaternary ammonium compound, which provides a microbicidal benefit, as compared to where such at least one such further detersive surfactant is absent, which is preferably at least one nonionic surfactant;

water;

optionally, one or more further constituents which impart one or more advantageous technical or aesthetic benefits to the compositions, including one or more detersive surfactants;

wherein the composition has a pH of at least 5,

wherein the surface treatment compositions are characterized in exhibiting a microbicidal benefit when tested according to one or more of the following standardized test protocols: ASTM E1052 Standard Test Method for Efficacy of Antimicrobial Agents against Viruses in Suspension, or ASTM E1053 Standard Test Method to Assess Virucidal Activity of Chemicals Intended for Disinfection of Inanimate, Nonporous Environmental Surfaces, or European Standard Surface Test, EN1369, or AOAC Germicidal Spray Products as Disinfectant Test Method, AOAC Index, 17^{th} Ed. (2000) against one or more challenge microorganisms, especially preferably against Poliovirus type 1 (Sabin) (“PV1”).

In a further aspect the present invention provides surface treatment compositions according to any foregoing aspects of the invention which compositions exhibit a pH of at least at least 6, yet more preferably at least 7.

It is to be understood that in each of the foregoing aspects, that the inanimate surface treatment compositions which impart may instead or also be used as air treatment compositions for a microbicidal benefit to treated air, particularly in a volume of air or headspace, e.g, in a closed room or the interior of a vehicle.

In a still further aspect the present invention provides a method of controlling the incidence of undesired microorganisms on an inanimate surface, the method comprising the step of: contacting the inanimate surface which is in need of treatment or upon which the presence of one or more undesirable microorganisms are suspected or are known to be present, with an effective amount of a liquid, inanimate surface treatment composition as described herein to provide a surface treatment benefit thereto, preferably to provide a microbicidal benefit to the contacted surface.

According to a further aspect of the present invention there is provided a method of controlling the incidence of undesired microorganisms in air, or in a headspace such as the ambient air within a closed volume such as a room or the interior of a vehicle, the method comprising the step of: delivering and dispersing within an airspace an effective amount of the liquid inanimate surface treatment composition as described herein to provide a microbicidal benefit to the treated air, preferably to provide a microbicidal benefit to the treated air.

In an additional aspect the present invention provides a vendible product, as well as a method for the manufacture of such a vendible product which comprises a surface treatment composition as described herein.

These and further aspects of the invention will become more apparent from a reading of the following specification.

A first essential constituent of the invention is a copper source material which releases copper ions into the treatment composition, preferably a source of Cu(I) and/or Cu(II) ions. The copper ions should be dispersible, miscible or soluble in the surface treatment compositions. Any material, or compound which may function as a source of copper ions, e.g., Cu(I) and/or Cu(II) ions which may deliver or provide such copper ions into an alcoholic liquid compositions, such as those described in this patent specification and particularly with reference to the examples, may be used in the present inventive compositions. Non-limiting examples of such materials or compounds include copper sulfate, copper chloride, copper nitrate, copper oxychloride, CuCl₂.2H₂O, Cu(OAcO)₂.H₂O, Cu D-gluconate, Cu(I)Cl.H₂O or any other chemical compound or chemical species which may be used to provide Cu(I) and especially Cu(II) ions into a largely alcoholic liquid composition. Such are to be expressly understood as non-limiting examples, and that other materials which may function to provide copper ions may be used, e.g., further copper containing salts of organic or inorganic compounds or materials. The copper ions need not be fully soluble within the largely alcoholic liquid compositions and may, for example, be dispersions. The copper source material may be present in the treatment compositions in any effective amount but advantageously is at least about 0.001% wt. to about 2.0% wt, preferably from about 0.01% wt to about 1% wt., and particularly preferably from about 0.01% wt. to about 0.5% wt. of the copper source material. Alternately, the copper source material may be present in the treatment compositions in a sufficient amount such that the copper source material releases copper ions into the treatment composition so to provide between about 1 ppm to about 10,000 ppm of Cu(I) and/or Cu(II) ions, preferably between about 20 ppm and about 5000 ppm of Cu(I) and/or Cu(II) ions, yet more preferably between about 50 ppm to about 1000 ppm of Cu(I) and/or Cu(II) ions, and particularly preferably between about 50 ppm to about 500 ppm of Cu(I) and/or Cu(II) ions within the inventive compositions taught herein.

Exclusive of counterions of surfactant compounds or counterions of other materials described herein which might be present, most preferably the copper source material is the sole material present in the composition which releases available metal ions to the treatment compositions taught herein.

A further essential constituent of the inventive compositions is at least one lower alkyl aliphatic monohydric alcohol. Preferably this at least one of a lower alkyl aliphatic monohydric alcohol also exhibits a biocidal effect against microorganisms independently of the other constituents which may be present in the compositions. Exemplary and preferred are C₁-C₆ mononhydric alcohols, especially methanol, ethanol, n-propanol, isopropanol, and all isomers of butanol. Of these, C₁-C₃ monohydric alcohols, and especially C₁-C₃mononhydric alcohols are preferred, especially ethanol. A single such alcohol, or mixture of two or more such alcohols may be present. In certain embodiments when a plurality of alcohols are present, ethanol is the predominant alcohol present, and especially preferably comprises at least 50.1% wt., and especially preferably and in order of increasing preference, at least 51%, 55%, 60%, 65%, 70%, 75%, 80%, 85%, 90%, 92%, 95%, 97%, 98%, 99%, 99.5% and 100% by weight of the at least one lower alkyl aliphatic monohydric alcohol present. In certain preferred embodiments, the at least one lower alkyl aliphatic monohydric alcohol comprises at least 20% wt of the treatment composition of which it forms a part. Preferably the at least one lower alkyl aliphatic monohydric alcohol constituent is present in the treatment composition in an amount of at least about 21% wt., and in order of increasing preference comprises at least 22%, 22.5%, 23%, 23.5%, 24%, 24.5%, 25%, 25.5%, 26%, 26.5%, 27%, 27.5% 28%, 28.5%, 29%, 29.5%, 30%, 30.5%, 31%, 31.5%, 32%, 32.5%, 33%, 33.5%, 34%, 34.5%, 35%, 35.5%, 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, 45%, 45.5%, 46%, 46.5%, 47%, 47.5%, 48%, 48.5%, 49%, 49.5%, 50%, 51%, 52%, 53%, 54%, 55%, 56%, 57%, 58%, 59%, 60% 61%, 62%, 63%, 64%, 65%, 66%, 67%, 68%, 69% and 70% by weight. Concurrently and preferably the at least one lower alkyl aliphatic monohydric alcohol constituent is present in the treatment composition in an amount of up to about 95% wt., and in order of increasing preference is present in an amount up to 85%, 80%, 75%, 70%, 69%, 68%, 67%, 66%, 65%, 64%, 63%, 62%, 61%, 60%, 59%, 58%, 57%, 56%, 55%, 54%, 53%, 52%, 51%, 50%, 49.5%, 49%, 48.5%, 48%, 47.5%, 47%, 46.5%, 46%, 45.5%, 45%, 44.5% 44%, 43.5%, 43%, 42.5%, 42%, 41.5%, 41%, 40.5%, 40%, 39.5%, 39%, 38.5%, 385, 37.5%, 37%, 36.5%, 36%, 35.5%, 35%, 34.5%, 34%, 33.5%, 33%, 32.5%, 32%, 31.5%, 31%, 30.5%, 30%, 29.5%, 29%, 28.5%, 28%, 27.5%, 27%, 26.5%, 26%, 25.5%, 25%, 24.5%, 24%, 23.5%, 23%, 22.5%, 22% , 21.5% and 21% by weight of the treatment composition of which it forms a part.

Advantageously the at least one at least one lower alkyl aliphatic monohydric alcohol constituent is one which exhibits a microbicidal effect against one or more pathogens even in the absence of the further constituents of the treatment compositions taught herein. For this reason, C₁-C₄ monohydric aliphatic alcohols, e.g., methanol, ethanol and the various isomers of propanol are particularly preferred whether used singly or in mixtures of two or more selected C₁-C₄ monohydric aliphatic alcohols. In certain embodiments a single C₁-C₄ monohydric aliphatic alcohol is present as the second essential constituent. In certain embodiments, ethanol is the sole constituent of the lower alkyl aliphatic monohydric alcohol constituent.

In certain preferred embodiments the compositions comprise from 20% wt. to 40% wt. of at least one lower alkyl aliphatic monohydric alcohol, and preferably in an amount up to but excluding 40% wt.

Water is the third essential constituent of the invention. The water may be tap water, but is preferably distilled and is most preferably deionized water. If the water is tap water, it is preferably substantially free of any undesirable impurities such as organics or inorganics, especially minerals salts which are present in hard water and which may undesirably interfere with the operation of the constituents present in the aqueous compositions according to the invention. Water is added to order to provide to 100% by weight of the compositions of the invention. Water comprises up to 80% wt. of the treatment compositions taught herein, although may be present in lesser amounts due to the inclusion of the further essential constituents, and optional constituents which may be present.

The present inventors have found that these three essential constituents, when at a pH of 5 or greater, preferably 6 or greater, form a highly effective system which exhibits a high degree of microbicidal efficacy even in the absence of further constituents. Advantageously however this system may be supplemented by one or more further constituents.

While not wishing to be bound by the following, the present inventors have surprisingly found that by careful selection of: (1) the nature and amounts of the copper source material which releases copper ions into the treatment composition, and especially preferably wherein the copper source material is a source of Cu(I) and/or Cu(II) ions, (2) the at least one aliphatic monohydric alcohol which itself exhibits an independent microbicidal effect, (3) water, and (4) the pH level of the surface treatment composition, the resultant compositions provide unexpectedly excellent microbicidal efficacy against a range of undesirable microorganisms including certain viruses, bacteria and certain fungi, which has heretofore not been expected from compositions which include the reduced amounts of the alcohol constituent provided in the inventive compositions. The resultant compositions provide unexpectedly excellent microbicidal efficacy against a range of undesirable microorganisms including certain viruses, bacteria and certain fungi, which has heretofore not been expected from compositions which include the reduced amounts of the alcohol constituent provided in the inventive compositions. Such an effect has been observed even when a very limited amount of the copper source material is present, and wherein the amount of the one lower alkyl aliphatic monohydric alcohol is also present in reduced amounts, e.g, in amount of between 20% wt. to about 50% wt, or even lesser amounts. Reference is made to the various Examples provided in this patent specification which demonstrates this effect, particularly as against comparative formulations which omit one or more of the copper source material, or the at least one alcohol. Although the literature describe the biochemical mechanisms of the separate microbicidal actives (the first, and the second essential constituents) when these microbicidal actives are used singly, surface treatment compositions as now disclosed by the inventors are believed to be unknown, particularly wherein such surface treatment compositions exhibit what is believed to be a synergistic benefit.

In a further aspect of the invention there is provided a microbicidal control system of constituents which are in and of themselves effective in providing effective control of poliovirus independently of further and optional constituents. This first microbicidal control system of constituents comprises (or consists essentially of, or consists of): water, a copper source material which releases copper ions into the treatment composition, one or more one or more C₁-C₄ aliphatic alcohols in an amount of up to, but less than 20% wt., and especially preferably wherein ethanol is the predominant or sole C₁-C₄ aliphatic alcohols present, a cationic quaternary ammonium compound and, where necessary, a buffer or pH adjusting agent to impart an alkaline pH, preferably an alkaline pH of 7.5 or greater. This microbicidal control system of constituents may thereafter optionally include further constituents which may or may not provide a further microbicidal benefit, such as a detersive surfactant, preferably a nonionic surfactant. In preferred embodiments, the microbial control system further includes least one further detersive surfactant, other than a quaternary ammonium surfactant compound which provides a microbicidal benefit, which further detersive surfactant is preferably at least one nonionic surfactant. Preferably the microbicidal control system is characterized in exhibiting a microbicidal benefit when tested against one or more challenge microorganisms according to one or more of the following standardized test protocols: ASTM E1052 Standard Test Method for Efficacy of Antimicrobial Agents against Viruses in Suspension, or ASTM E1053 Standard Test Method to Assess Virucidal Activity of Chemicals Intended for Disinfection of Inanimate, Nonporous Environmental Surfaces, or European Standard Surface Test, EN1369, or AOAC Germicidal Spray Products as Disinfectant Test Method, AOAC Index, 17^{th} Ed. (2000), especially preferably against poliovirus type 1 (Sabin) (“PV1”).

Further aspects of the invention include surface treatment compositions which comprise a microbicidal control system of constituents as described above.

The surface treatment compositions may further optionally comprise one or more additional constituents which impart one or more advantageous technical or aesthetic benefits to the compositions.

Optionally but in many cases, preferably the treatment compositions also comprise at least one quaternary ammonium compound which provides a microbicidal benefit. For the purposes of the present invention described herein, such quaternary ammonium compounds are to be understood as being outside of the scope of the defined detersive surfactants as such materials are primarily provided to impart a microbicidal effect, and not provide an appreciable detersive benefit. Any cationic surfactant which satisfies these requirements may be used and is considered to be within the scope of the present invention. Mixtures of two or more cationic surface active agents, viz., cationic surfactants may also be used. Cationic surfactants are well known, and useful cationic surfactants may be one or more of those described for example in McCutcheon's Functional Materials, Vol.2, 1998; Kirk-Othmer, Encyclopedia of Chemical Technology, 4th Ed., Vol. 23, pp. 481-541 (1997), the contents of which are herein incorporated by reference. These are also described in the respective product specifications and literature available from the suppliers of these cationic surfactants.

Examples of preferred cationic surfactant compositions useful in the practice of the instant invention are those which provide a microbicidal or germicidal effect to the compositions, and especially preferred are quaternary ammonium compounds and salts thereof, which may be characterized by the general structural formula:

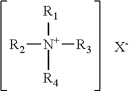

where at least one of R₁, R₂, R₃ and R₄ is a alkyl, aryl or alkylaryl substituent of from 6 to 26 carbon atoms, and the entire cation portion of the molecule has a molecular weight of at least 165. The alkyl substituents may be long-chain alkyl, long-chain alkoxyaryl, long-chain alkylaryl, halogen-substituted long-chain alkylaryl, long-chain alkylphenoxyalkyl, arylalkyl, etc. The remaining substituents on the nitrogen atoms other than the abovementioned alkyl substituents are hydrocarbons usually containing no more than 12 carbon atoms. The substituents R₁, R₂, R₃ and R₄ may be straight-chained or may be branched, but are preferably straight-chained, and may include one or more amide, ether or ester linkages. The counterion X may be any salt-forming anion which permits for the solubility of the quaternary ammonium complex within the treatment composition.

Exemplary quaternary ammonium salts within the above description include the alkyl ammonium halides such as cetyl trimethyl ammonium bromide, alkyl aryl ammonium halides such as octadecyl dimethyl benzyl ammonium bromide, N-alkyl pyridinium halides such as N-cetyl pyridinium bromide, and the like. Other suitable types of quaternary ammonium salts include those in which the molecule contains either amide, ether or ester linkages such as octyl phenoxy ethoxy ethyl dimethyl benzyl ammonium chloride, N-(laurylcocoaminoformylmethyl)-pyridinium chloride, and the like. Other very effective types of quaternary ammonium compounds which are useful as germicides include those in which the hydrophobic radical is characterized by a substituted aromatic nucleus as in the case of lauryloxyphenyltrimethyl ammonium chloride, cetylaminophenyltrimethyl ammonium methosulfate, dodecylphenyltrimethyl ammonium methosulfate, dodecylbenzyltrimethyl ammonium chloride, chlorinated dodecylbenzyltrimethyl ammonium chloride, and the like.

Preferred quaternary ammonium compounds which act as germicides and which are useful in the practice of the present invention include those which have the structural formula:

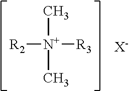

wherein R₂ and R₃ are the same or different C₈-C₁₂alkyl, or R₂ is C₁₂₋₁₆alkyl, C₈₋₁₈alkylethoxy, C₈₋₁₈alkylphenolethoxy and R₃ is benzyl, and X is a halide, for example chloride, bromide or iodide, a saccharinate counterion or is a methosulfate anion. The alkyl groups recited in R₂ and R₃ may be straight-chained or branched, but are preferably substantially linear.

Particularly useful quaternary ammonium compounds include compositions which include a single quaternary compound, as well as mixtures of two or more different quaternary compounds. Such useful quaternary compounds are available under the BARDAC®, BARQUAT®, HYAMINE®, LONZABAC®, and ONYXIDE® trademarks, which are more fully described in, for example, McCutcheon's Functional Materials (Vol. 2), North American Edition, 1998, as well as the respective product literature from the suppliers identified below. Such include, for example, BARDAC® 205M which is described to be a liquid containing alkyl dimethyl benzyl ammonium chloride, octyl decyl dimethyl ammonium chloride; didecyl dimethyl ammonium chloride, and dioctyl dimethyl ammonium chloride (50% active) (also available as 80% active (BARDAC® 208M)); BARDAC® 2050 which is described to be a combination of octyl decyl dimethyl ammonium chloride/didecyl dimethyl ammonium chloride, and dioctyl dimethyl ammonium chloride (50% active) (also available as 80% active (BARDAC® 2080)); BARDAC® 2250 which is described to be didecyl dimethyl ammonium chloride (50% active); BARDAC® LF (or BARDAC® LF-80), described as being based on dioctyl dimethyl ammonium chloride (BARQUAT® MB-50, MX-50, OJ-50 (each 50% liquid) and MB-80 or MX-80 (each 80% liquid) are each described as an alkyl dimethyl benzyl ammonium chloride; BARDAC® 4250 and BARQUAT® 4250Z (each 50% active) or BARQUAT® 4280 and BARQUAT 4280Z (each 80% active) are each described as alkyl dimethyl benzyl ammonium chloride/alkyl dimethyl ethyl benzyl ammonium chloride. Also, HYAMINE® 1622, described as diisobutyl phenoxy ethoxy ethyl dimethyl benzyl ammonium chloride (50% solution); HYAMINE® 3500 (50% actives), described as alkyl dimethyl benzyl ammonium chloride (also available as 80% active (HYAMINE® 3500-80)); and HYMAINE® 2389 described as being based on methyldodecylbenzyl ammonium chloride and/or methyldodecylxylene-bis-trimethyl ammonium chloride. (BARDAC®, BARQUAT® and HYAMINE® are presently commercially available from Lonza, Inc., Fairlawn, N.J.). BTC® 50 NF (or BTC® 65 NF) is described to be alkyl dimethyl benzyl ammonium chloride (50% active); BTC® 99 is described as didecyl dimethyl ammonium chloride (50% active); BTC® 776 is described to be myrisalkonium chloride (50% active); BTC® 818 is described as being octyl decyl dimethyl ammonium chloride, didecyl dimethyl ammonium chloride, and dioctyl dimethyl ammonium chloride (50% active) (available also as 80% active (BTC® 818-80%)); BTC® 824 and BTC® 835 are each described as being of alkyl dimethyl benzyl ammonium chloride (each 50% active); BTC® 885 is described as a combination of BTC® 835 and BTC® 818 (50% active) (available also as 80% active (BTC® 888)); BTC® 1010 is described as didecyl dimethyl ammonium chloride (50% active) (also available as 80% active (BTC® 1010-80)); BTC® 2125 (or BTC® 2125 M) is described as alkyl dimethyl benzyl ammonium chloride and alkyl dimethyl ethylbenzyl ammonium chloride (each 50% active) (also available as 80% active (BTC® 2125 80 or BTC® 2125 M)); BTC® 2565 is described as alkyl dimethyl benzyl ammonium chlorides (50% active) (also available as 80% active (BTC® 2568)); BTC® 8248 (or BTC® 8358) is described as alkyl dimethyl benzyl ammonium chloride (80% active) (also available as 90% active (BTC® 8249)); ONYXIDE® 3300 is described as n-alkyl dimethyl benzyl ammonium saccharinate (95% active). (BTC® and ONYXIDE® are presently commercially available from Stepan Company, Northfield, Ill.) Polymeric quaternary ammonium salts based on these monomeric structures are also considered desirable for the present invention. One example is POLYQUAT®, described as being a 2-butenyldimethyl ammonium chloride polymer.

When present, the quaternary ammonium compound(s) may be present in any effective amount, but generally need not be present in amounts in excess of about 10% wt. based on the total weight of the composition. Preferably the germicidal quaternary ammonium compounds may be present in the inventive compositions in amounts of from about 0.001% wt. to up to about 10% wt., very preferably about 0.01-8% wt., more preferably in amounts of between about 0.01-2% wt., and most preferably from about 0.01 - 1% wt. It is particularly advantageous that the preferred germicidal cationic surfactant(s) are present in amounts of at least about 200 parts per million (ppm), preferably in amounts of from about 1 ppm to about 10,000 ppm, preferably from about 50 ppm to about 2000 ppm, more preferably in amounts of from about 100 ppm to about 1,000 ppm. Particularly preferred amounts of one or more quaternary ammonium compound(s) and preferred amounts are identified with reference to the examples.

In certain preferred embodiments the treatment compositions necessarily include at least at least one further detersive surfactant, (which is preferably a nonionic surfactant) other than a germicidally effective quaternary ammonium compound, which least one further detersive surfactant provides a further microbicidal benefit within the treatment composition of which it forms a part, as compared to where such at least one such further detersive surfactant is absent from said composition.

In certain embodiments the treatment compositions necessarily include at least one further, detersive surfactant, although such may be considered an optional constituent according to other embodiments of the invention.

Non-limiting examples of the major surfactant types that can be used as detersive surfactants of the present invention include those which are known as anionic, nonionic, amphoteric, and zwitterionic surfactants as well as further cationic surfactants which are not primarily present to provide a microbicidal or germicidal benefit. Such include, e.g.: sulfates and sulfonates of oils and fatty acids, sulfates and sulfonates, ethoxylated alkylphenols, sulfates of alcohols, sulfates of ethoxylated alcohols, sulfates of fatty esters, sulfonates of benzene, cumene, toluene and xylene, sulfonates of condensed naphthalenes, sulfonates of dodecyl and tridecylbenzenes, sulfonates of naphthalene and alkyl naphthalene, sulfonates of petroleum, sulfosuccinamates, sulfosuccinates and derivatives, soaps, taurates, thio and mercapto derivatives, tridecyl and dodecyl benzene sulfonic acids, alkanolamides, alkanolamines, alkylaryl sulfonates, alkylaryl sulfonic acids, alkylbenzenes, amine acetates, amine oxides, amines, sulfonated amines and amides, betaine derivatives, block polymers, carboxylated alcohol or alkylphenol ethoxylates, carboxylic acids and fatty acids, ethoxylated alcohols, ethoxylated alkylphenols, ethoxylated amines and/or amides, ethoxylated fatty acids, ethoxylated fatty esters and oils, fatty esters, fluorocarbon-based surfactants, glycerol esters, glycol esters, hetocyclic-type products, imidazolines and imidazoline derivatives, isethionates, lanolin-based derivatives, lecithin and lecithin derivatives, lignin and lignin deriviatives, maleic or succinic anhydrides, methyl esters, monoglycerides and derivatives, olefin sulfonates, phosphate esters, phosphorous organic derivatives, polyethylene glycols, polymeric (polysaccharides, acrylic acid, and acrylamide) surfactants, propoxylated and ethoxylated fatty acid alcohols or alkyl phenols, protein-based surfactants, sarcosine derivatives, silicone-based surfactants, sorbitan derivatives, sucrose and glucose esters and derivatives, as well as further surfactants known to the art but not elucidated here.

Additional non-limiting examples of detersive surfactants that can be used to carry out the present invention include one or more nonionic surfactants, especially one or more compounds based on the condensation products of alkylene oxide groups with an organic hydrophobic compound, such as an aliphatic compound or with an alkyl aromatic compound. The nonionic synthetic organic detergents generally are the condensation products of an organic aliphatic or alkyl aromatic hydrophobic compound and hydrophilic ethylene oxide groups. Practically any hydrophobic compound having a carboxy, hydroxy, amido, or amino group with a free hydrogen attached to the nitrogen can be condensed with ethylene oxide or with the polyhydration product thereof, polyethylene glycol, to form a water soluble nonionic detergent. Further, the length of the polyethenoxy hydrophobic and hydrophilic elements may be varied to adjust these properties. Illustrative examples of such a nonionic surfactant include the condensation product of one mole of an alkyl phenol having an alkyl group containing from 6 to 12 carbon atoms with from about 5 to 25 moles of an alkylene oxide. Another example of such a nonionic surfactant is the condensation product of one mole of an aliphatic alcohol which may be a primary, secondary or tertiary alcohol having from 6 to 18 carbon atoms with from 1 to about 10 moles of alkylene oxide. Preferred alkylene oxides are ethylene oxides or propylene oxides which may be present singly, or may be both present.

Non-limiting, illustrative examples of nonionic surfactants include primary and secondary linear and branched alcohol ethoxylates, such as those based on C₆-C₁₈ alcohols which further include an average of from 2 to 80 moles of ethoxylation per mol of alcohol. Examples include the Genapol® series of linear alcohol ethoxylates from Clariant Corp., Charlotte, N.C. The 26-L series is based on the formula RO(CH₂CH₂O)ₙH wherein R is a mixture of linear, even carbon-number hydrocarbon chains ranging from C ₁₂H₂₅ to C₁₆H₃₃ and n represents the number of repeating units and is a number of from 1 to about 12, such as 26-L-1, 26-L-1.6, 26-L-2, 26-L-3, 26-L-5, 26-L-45, 26-L-50, 26-L-60, 26-L-60N, 26-L-75, 26-L-80, 26-L-98N, and the 24-L series, derived from synthetic sources and typically contain about 55% C₁₂ and 45% C₁₄ alcohols, such as 24-L-3, 24-L-45, 24-L-50, 24-L-60, 24-L-60N, 24-L-75, 24-L-92, and 24-L-98N. From product literature, the single number following the “L” corresponds to the average degree of ethoxylation (numbers between 1 and 5) and the two digit number following the letter “L” corresponds to the cloud point in ° C. of a 1.0 wt. % solution in water.

Further examples of useful nonionic surfactants include secondary C ₁₂-C₁₅ alcohol ethoxylates, including those which have from about 3 to about 10 moles of ethoxylation. Such are available in the Tergitol® series of nonionic surfactants (Dow Chemical, Midland, Mich.), particularly those in the Tergitol® “15-S-” series. Further exemplary nonionic surfactants include linear primary C₁₁-C₁₅ alcohol ethoxylates, including those which have from about 3 to about 10 moles of ethoxylation. Such are available in the Tomadol® series of nonionic surfactants under the following tradenames: Tomadol 1-3 (linear C₁₁ alcohol with 3 moles (average) of ethylene oxide); Tomadol 1-5 (linear C₁₁ alcohol with 5 moles (average) of ethylene oxide); Tomadol 1-7 (linear C₁₁ alcohol with 7 moles (average) of ethylene oxide); Tomadol 1-9 (linear C₁₁ alcohol with 9 moles (average) of ethylene oxide); Tomadol 23-1 (linear C₁₂₋₁₃ alcohol with 1 mole (average) of ethylene oxide); Tomadol 23-3 (linear C₁₂₋₁₃ alcohol with 3 moles (average) of ethylene oxide); Tomadol 23-5 (linear C₁₂₋₁₃ alcohol with 5 moles (average) of ethylene oxide); Tomadol 23-6.5 (linear C₁₂₋₁₃ alcohol with 6.6 moles (average) of ethylene oxide); Tomadol 25-12 (linear C₁₂₋₁₅ alcohol with 11.9 moles (average) of ethylene oxide); Tomadol 25-3 (linear C₁₂₋₁₅ alcohol with 2.8 moles (average) of ethylene oxide); Tomadol 25-7 (linear C₁₂₋₁₅ alcohol with 7.3 moles (average) of ethylene oxide); Tomadol 25-9 (linear C₁₂₋₁₅ alcohol with 8.9 moles (average) of ethylene oxide); Tomadol 45-13 (linear C₁₄₋₁₅ alcohol with 12.9 moles (average) of ethylene oxide); Tomadol 45-2.25 (linear C₁₄₋₁₅ alcohol with 2.23 moles (average) of ethylene oxide); Tomadol 45-7 (linear C₁₄₋₁₅ alcohol with 7 moles (average) of ethylene oxide); Tomadol 91-2.5 (linear C₉₋₁₁ alcohol with 2.7 moles (average) of ethylene oxide); Tomadol 91-6 (linear C₉₋₁₁ alcohol with 6 moles (average) of ethylene oxide); Tomadol 91-8 (linear C₉₋₁₁ alcohol with 8.3 moles (average) of ethylene oxide) (Tomah Products, Inc., Milton, Wis.).

Further examples of useful nonionic surfactants include C₆-C₁₅ straight chain alcohols ethoxylated with about 1 to 13 moles of ethylene oxide, particularly those which include about 3 to about 6 moles of ethylene oxide. Examples of such nonionic surfactants include Alfonic® 810-4.5, which is described as having an average molecular weight of 356, an ethylene oxide content of about 4.85 moles and an HLB of about 12; Alfonic® 810-2, which is described as having an average molecular weight of 242, an ethylene oxide content of about 2.1 moles and an HLB of about 12; and Alfonic® 610-3.5, which is described as having an average molecular weight of 276, an ethylene oxide content of about 3.1 moles, and an HLB of 10.

A further class of nonionic surfactants which may find use in the present inventive compositions include ethoxylated octyl and nonyl phenols include those having one of the following general structural formulas:

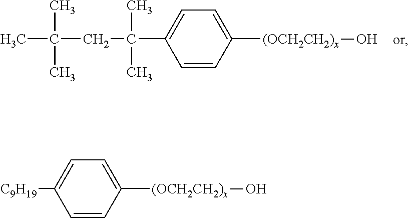

in which the C₉H₁₉ group in the latter formula is a mixture of branched chained isomers, and x indicates an average number of ethoxy units in the side chain. Particularly suitable non-ionic ethoxylated octyl and nonyl phenols include those having from about 7 to about 13 ethoxy groups. Such compounds are commercially available under the trade name Triton® X (Dow Chemical, Midland, Mich.), as well as under the tradename Igepal® (Rhodia, Princeton, N.J.). One such exemplary and particularly preferred surfactant is nonylphenol ethoxylate is Igepal® CO-630.

Still further examples of suitable nonionic surfactants include which may be advantageously included in the inventive compositions are alkoxy block copolymers, and in particular, compounds based on ethoxy/propoxy block copolymers. Polymeric alkylene oxide block copolymers include nonionic surfactants in which the major portion of the molecule is made up of block polymeric C2-C4 alkylene oxides. Such nonionic surfactants, while preferably built up from an alkylene oxide chain starting group, and can have as a starting nucleus almost any active hydrogen containing group including, without limitation, amides, phenols, thiols and secondary alcohols.

One group of such useful nonionic surfactants containing the characteristic alkylene oxide blocks are those which may be generally represented by the formula (A):

HO—(EO)ₓ(PO)_{y}(EO)_{z}-H (A)

where EO represents ethylene oxide,

PO represents propylene oxide,

y equals at least 15,

(EO)_{x+Z} equals 20 to 50% of the total weight of said compounds, and, the total molecular weight is preferably in the range of about 2000 to 15,000.

Another group of nonionic surfactants for use in the new compositions can be represented by the formula (B):

R—(EO,PO)ₐ(EO,PO)_{b}—H (B)

wherein R is an alkyl, aryl or aralkyl group, where the R group contains 1 to 20 carbon atoms, the weight percent of EO is within the range of 0 to 45% in one of the blocks a, b, and within the range of 60 to 100% in the other of the blocks a, b, and the total number of moles of combined EO and PO is in the range of 6 to 125 moles, with 1 to 50 moles in the PO rich block and 5 to 100 moles in the EO rich block.

Further nonionic surfactants which in general are encompassed by formula (B) include butoxy derivatives of propylene oxide/ethylene oxide block polymers having molecular weights within the range of about 2000-5000.

Still further useful nonionic surfactants containing polymeric butoxy (BO) groups can be represented by formula (C) as follows:

RO—(BO)ₙ(EO)ₓ-H (C)

wherein R is an alkyl group containing 1 to 20 carbon atoms,

n is about 5-15 and x is about 5-15.

Also useful as the nonionic block copolymer surfactants, which also include polymeric butoxy groups, are those which may be represented by the following formula (D):

HO—(EO)ₓ(BO)ₙ(EO)_{y}-H (D)

wherein n is about 5-15, preferably about 15,

x is about 5-15, preferably about 15, and

y is about 5-15, preferably about 15.

Still further useful nonionic surfactants include ethoxylated derivatives of propoxylated ethylene diamine, which may be represented by the following formula:

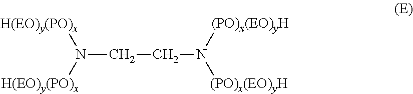

where (EO) represents ethoxy,

(PO) represents propoxy,

the amount of (PO)ₓ is such as to provide a molecular weight prior to ethoxylation of about 300 to 7500, and the amount of (EO)_{y} is such as to provide about 20% to 90% of the total weight of said compound.

Further examples of useful nonionic surfactants are one or more amine oxides. Exemplary amine oxides include:

A) Alkyl di (lower alkyl) amine oxides in which the alkyl group has about 10-20, and preferably 12-16 carbon atoms, and can be straight or branched chain, saturated or unsaturated. The lower alkyl groups include between 1 and 7 carbon atoms. Examples include lauryl dimethyl amine oxide, myristyl dimethyl amine oxide, and those in which the alkyl group is a mixture of different amine oxide, dimethyl cocoamine oxide, dimethyl (hydrogenated tallow) amine oxide, and myristyl/palmityl dimethyl amine oxide;

B) Alkyl di (hydroxy lower alkyl) amine oxides in which the alkyl group has about 10-20, and preferably 12-16 carbon atoms, and can be straight or branched chain, saturated or unsaturated. Examples are bis(2-hydroxyethyl) cocoamine oxide, bis(2-hydroxyethyl) tallowamine oxide; and bis(2-hydroxyethyl) stearylamine oxide;

C) Alkylamidopropyl di(lower alkyl) amine oxides in which the alkyl group has about 10-20, and preferably 12-16 carbon atoms, and can be straight or branched chain, saturated or unsaturated. Examples are cocoamidopropyl dimethyl amine oxide and tallowamidopropyl dimethyl amine oxide; and

D) Alkylmorpholine oxides in which the alkyl group has about 10-20, and preferably 12-16 carbon atoms, and can be straight or branched chain, saturated or unsaturated.

Preferably the amine oxide constituent is an alkyl di (lower alkyl) amine oxide as denoted above and which may be represented by the following structure:

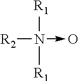

wherein each:

R₁ is a straight chained C₁-C₄ alkyl group, preferably both R₁ are methyl groups; and,

R₂ is a straight chained C₈-C₁₈ alkyl group, preferably is C₁₀-C₁₄ alkyl group, most preferably is a C₁₂ alkyl group.

Each of the alkyl groups may be linear or branched, but most preferably are linear. Technical grade mixtures of two or more amine oxides may be used, wherein amine oxides of varying chains of the R₂ group are present. Preferably, the amine oxides used in the present invention include R₂ groups which comprise at least 50% wt., preferably at least 60% wt. of C₁₂ alkyl groups and at least 25% wt. of C₁₄ alkyl groups, with not more than 15% wt. of C₁₆, C₁₈ or higher alkyl groups as the R₂ group.

Further specific examples of useful nonionic surfactants are alkanolamide surfactant compounds. Exemplary useful alkanolamides include one or more monoethanol amides, and diethanol amides of fatty acids having an acyl moiety which contains from about 8 to about 18 carbon atoms, and which may be represented in accordance with the formula:

R₁—CO—N(H)ₘ₋₁(R₂OH)₃₋ₘ

where R₁ represents a saturated or unsaturated aliphatic hydrocarbon radical of from about 7 to 21 carbon atoms, but preferably from about 11 to 17 carbon atoms; R₂ represents a —CH₂— or —CH₂CH₂—, and m is an integer from 1 to 3, but is preferably 1. Preferably, R₁ is a saturated or unsaturated aliphatic hydrocarbon radical comprising from about 11 to 17 carbon atoms, and m is 1. Specific examples of such compounds include mono-ethanol amine coconut fatty acid amide and diethanol amine dodecyl fatty acid amide. An exemplary useful and particularly preferred fatty acid amides include cocomonoethanol amide or cocodiethanolamide, which are presently commercially available under the Monamid® tradename. Further exemplary useful alkanolamides which provide such functions include inter alia: cocamide MEA, cocamide DEA, soyamide DEA, lauramide DEA, oleamide MIPA, stearamide MEA, myristamide MEA, lauramide MEA, capramide DEA, ricinoleamide DEA, myristamide DEA, stearamide DEA, oleylamide DEA, tallowamide DEA, lauramide MIPA, tallowamide MEA, isostearamide DEA, isostearamide MEA, and mixtures thereof. Further useful alkanolamide surfactant compounds include alkanolamides, particularly fatty monoalkanolamides and fatty dialkanolamides, including one or more of those marketed under the Nino® tradename. Further exemplary alkanolamide surfactant compounds include monoethanol amides and diethanol amides include those marketed under the trade names Alakamide® and Cyclomide® by Rhone-Poulenc Co., (Cranbury, N.J.) e.g., Cyclomide® CDD-518 described to be a nonionic surfactant based on coconut diethanolamide; Cyclomide® C212 described to be a nonionic surfactant based on coconut monoethanolamide; Cyclomide® DC212/SE described to be a nonionic surfactant based on 1:1 fatty acid diethanolamide; Cyclomide® DIN 100 described to be a nonionic surfactant based on lauric/linoleic diethanolamide; Cyclomide® DIN-295/S described to be a nonionic surfactant based on 1:1 linoleic diethanolamide; Cyclomide® DL203 described to be a nonionic surfactant based on 2:1 lauric diethanolamide.

Further specific examples of useful nonionic surfactants include alkyl polyglycosides. The alkyl polyglycosides which can be used as nonionic surfactants in the composition are generally represented by the formula:

R₁O(R₂O)_{b}(Z)ₐ

wherein R₁ is a monovalent organic radical having from about 6 to about 30 carbon atoms; R₂ is a divalent alkylene radical having from 2 to 4 carbon atoms; Z is a saccharide residue having 5 or 6 carbon atoms; b is a number having a value from 0 to about 12; a is a number having a value from 1 to about 6. Preferred alkyl polyglycosides have the formula I wherein Z is a glucose residue and b is zero. Such alkyl polyglycosides are commercially available, for example, as APG®, GLUCOPON®, or PLANTAREN® surfactants from Cogis Corp. Specific examples of such surfactants include but are not limited to: APG® 225, described to be an alkyl polyglycoside in which the alkyl group contains 8 to 10 carbon atoms and having an average degree of polymerization of 1.7; GLUCOPON® 425, described to be an alkyl polyglycoside in which the alkyl group contains 8 to 16 carbon atoms and having an average degree of polymerization of 1.48.; GLUCOPON® 625, described to be an alkyl polyglycoside in which the alkyl group contains 12 to 16 carbon atoms and having an average degree of polymerization of 1.6; APG® 325, described to be an alkyl polyglycoside in which the alkyl group contains 9 to 11 carbon atoms and having an average degree of polymerization of 1.5; GLUCOPON® 600, described to be an alkyl polyglycoside in which the alkyl group contains 12 to 16 carbon atoms and having an average degree of polymerization of 1.4; PLANTAREN® 2000, described to be an alkyl polyglycoside in which the alkyl group contains 8 to 16 carbon atoms and having an average degree of polymerization of 1.4; and, PLANTAREN® 1300, described to be an alkyl polyglycoside in which the alkyl group contains 12 to 16 carbon atoms and having an average degree of polymerization of 1.6. Other examples include alkyl polyglycoside surfactant compositions which are comprised of mixtures of compounds of formula I wherein Z represents a moiety derived from a reducing saccharide containing 5 or 6 carbon atoms; a is a number having a value from 1 to about 6; b is zero; and R.sub.1 is an alkyl radical having from 8 to 20 carbon atoms. The compositions are characterized in that they have increased surfactant properties and an HLB in the range of about 10 to about 16 and a non-Flory distribution of glycosides, which is comprised of a mixture of an alkyl monoglycoside and a mixture of alkyl polyglycosides having varying degrees of polymerization of 2 and higher in progressively decreasing amounts, in which the amount by weight of polyglycoside having a degree of polymerization of 2, or mixtures thereof with the polyglycoside having a degree of polymerization of 3, predominate in relation to the amount of monoglycoside, said composition having an average degree of polymerization of about 1.8 to about 3. Such compositions, also known as peaked alkyl polyglycosides, can be prepared by separation of the monoglycoside from the original reaction mixture of alkyl monoglycoside and alkyl polyglycosides after removal of the alcohol. This separation may be carried out by molecular distillation and normally results in the removal of about 70-95% by weight of the alkyl monoglycosides. After removal of the alkyl monoglycosides, the relative distribution of the various components, mono- and poly-glycosides, in the resulting product changes and the concentration in the product of the polyglycosides relative to the monoglycoside increases as well as the concentration of individual polyglycosides to the total, i.e. DP2 and DP3 fractions in relation to the sum of all DP fractions. Such compositions are disclosed in U.S. Pat. No. 5,266,690, the entire contents of which are incorporated herein by reference.

Other alkyl polyglycosides which can be used in the compositions according to the invention are those in which the alkyl moiety contains from 6 to 18 carbon atoms in which and the average carbon chain length of the composition is from about 9 to about 14 comprising a mixture of two or more of at least binary components of alkylpolyglycosides, wherein each binary component is present in the mixture in relation to its average carbon chain length in an amount effective to provide the surfactant composition with the average carbon chain length of about 9 to about 14 and wherein at least one, or both binary components, comprise a Flory distribution of polyglycosides derived from an acid-catalyzed reaction of an alcohol containing 6-20 carbon atoms and a suitable saccharide from which excess alcohol has been separated.

Also useful as nonionic surfactants are ethylene oxides condensed with sorbitan fatty acid esters. Such materials are presently commercially available under the tradename TWEEN (ex. ICI) and/or CRILL (ex. Croda) which include polyoxyethylene sorbitan monolaurate, polyoxyethylene sorbitan monopalmitate, polyoxyethylene sorbitan monostearate, polyoxyethylene sorbitan tristearate, polyoxyethylene sorbitan monooleate, polyoxyethylene sorbitan trioleates which are available in a variety of grades, and with differing amounts of polyoxylethylene groups per molecule.

The inventive compositions most desirably, although not always essentially, include at least one nonionic surfactant. An example of an especially preferred nonionic surfactant is at least one alcohol ethoxylate based nonionic surfactant in an amount of from about 0.01-10% wt. In order of increasing preference, when present, the at least one nonionic surfactant comprises in % wt. at least 0.025, 0.05, 0.075, 0.1, 0.2, 0.25, 0.3, 0.4, 0.5, 0.6, 0.75, 0.8, 0.9, 1, 1.1, 1.2, 1.3, 1.4 and 1.5% wt. and similarly in order of increasing preference the at least one nonionic surfactant comprises, in % wt., not more than 10, 9, 8, 7.5, 7, 6, 5, 4.75, 4.5, 4, 3.75, 3.5, 3.25, 3, 2.75 and 2% wt. based on the total weight of a treatment composition of which they form a part.

Especially preferred nonionic surfactants and the amounts in which they are preferably present are disclosed with reference to one or more of the Examples. In certain embodiments at least one nonionic surfactant is necessarily present and is considered as a further essential constituent of the invention.

Non-limiting examples of further detersive surfactants which may be included in the treatment compositions of the invention include zwitterionic and amphoteric surfactants. Zwitterionic surfactants may also be present either by themselves or in admixture with another ionic surfactant providing there are no troublesome interactions. Typical examples of amphoteric or zwitterionic surfactants are alkyl betaines, alkyl amidobetaines, aminopropionates, aminoglycinates, imidazolinium betaines and sulfobetaines. Within this group, alkyl betaines and alkyl amidobetaines are particularly preferred. Alkyl betaines are known surfactants which are mainly produced by carboxyalkylation, preferably carboxymethylation of aminic compounds. Typical examples are the carboxymethylation products of hexyl methyl amine, hexyl dimethyl amine, octyl dimethyl amine, decyl dimethyl amine, dodecyl methyl amine, dodecyl dimethyl amine, dodecyl ethyl methyl amine, C12/14 cocoalkyl dimethyl amine, myristyl dimethyl amine, cetyl dimethyl amine, stearyl dimethyl amine, stearyl ethyl methyl amine, oleyl dimethyl amine, C16/18 tallow alkyl dimethyl amine and technical mixtures thereof.

Alkyl amidobetaines which represent carboxyalkylation products of amidoamines are also suitable. Typical examples are reaction products of fatty acids containing 6 to 22 carbon atoms, namely caproic acid, caprylic acid, capric acid, lauric acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof, with N,N-dimethylaminoethyl amine, N,N-dimethylaminoproply amine, N,N-diethylaminoethyl amine and N,N-diethylaminoproply amine which are condensed with sodium chloroacetate. The condensation product of C8/18 cocofatty acid-N,N-dimethylaminopropyl amide with sodium chloroacetate is preferably used.

Further specific examples of particular amphoteric surfactants which may be used in the treatment compositions of the invention include one or more amphoteric surfactants. Exemplary amphoteric surfactants include alkylampho(mono)acetates, alkylampho(di)acetates, alkylampho(mono)propionates, and alkylampho(di)propionates. Examples of these amphoteric surfactants can be found under the tradename Miranol from Rhodia (Cranbury, N.J.). Some examples include Miranol C2M-Conc. NP, described to be disodium cocoamphodiacetate; Miranol FA-NP, described to be sodium cocoamphotacetate; Miranol DM, described to be sodium steroamphoacetate; Miranol HMA, described to be sodium lauroamphoacetate; Miranol C2M, described to be cocoamphodiprioponic acid; Miranol C2M-SF, described to be disodium cocoamphodiproprionate; Miranol CM-SF Conc., described as being cocoamphopropriate; Mirataine H2C-HA, described as sodium lauiminodiproprionate; Miranol Ultra L-32, described as sodium lauroamphoacetate; and Miranol Ultra C-37, described as sodium cocoamphoacetate. Other amphoteric surfactants are also available under the tradename Amphoterge from Lonza (Fair Lawn, N.J.) such as Amphoterge K described to sodium cocoamphoproprionate; Amphoterge K-2, described as disodium cocoamphodiproprionate; Amphoterge W, described to be sodium cocoamphoacetate; and Amphoterge W-2, described to be disodium cocoamphodiacetate.

Further useful amphoteric surfactants include those which may be represented by the following general formula

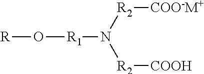

in which, R represents a C₄ to C₂₄ alkyl group, and is preferably a C10 to C16 alkyl group, R1 and R2 independently represent a C₁ to C₈ alkyl group, is preferably —CH₂CH₂— or —CH₂CH₂CH₂—, and M may be any salt-forming anion which permits water solubility or water miscibility of the compound, e.g., chloride, bromide, methosulfate, ethosulfate, lactate, saccharinate, acetate or phosphate. Such compounds are presently commercially available, such as those marketed in the Tomamine Amphoteric series of amphoteric surfactants, ex. Air Products Inc.

While the one or more detersive surfactants may be present in any effective amount which may be observed to improve the microbicidal efficacy of the system of the essential constituents, these one or more surfactants, when present, are advantageously present in an amount of from about 0.001-15% wt., preferably from about 0.01-10% wt. and particularly preferably from about 0.05-5% wt., based on the total weight of the treatment composition within which they are present.

In certain preferred embodiments, at least one detersive surfactant, preferably at least one nonionic surfactant, is a necessary constituent of the inventive compositions. While not wishing to be bound by the following, it is suspected that the presence of at least one surfactant and especially at least one nonionic surfactant aids in the penetration of organic soils and/or the penetration of one or more undesired microorganisms and hastens the activity of the essential constituents, viz. the copper ions provided by the source of copper ions, and/or the lower alkyl aliphatic monohydric alcohol and/or at least one quaternary ammonium compound which provides a microbicidal benefit in reducing, deactivating or destroying these undesired microorganisms and thus may aid in both providing an improved speed and/or degree of control, reduction or elimination of the one or more undesired microorganisms being treated with the treatment compositions taught herein.

In certain embodiments, the sole surfactants present in the compositions are the at least one or more of the cationic surfactants described above, and/or one or more of the nonionic surfactants described above. In certain embodiments the sole surfactant(s) present is one or more cationic surfactants. In certain embodiments the sole surfactant(s) present is one or more nonionic surfactants.

The pH of the treatment compositions is preferably established and thereafter maintained at a desired pH or within a bounded pH range. As is better understood from a consideration of the example compositions, the inventors have also found that the pH of the treatment compositions plays a significant role in establishing the overall efficacy of a treatment composition in reducing, deactivating or destroying undesired microorganisms. It was generally, observed that compositions having a higher, more alkaline pH but concurrently including lesser amounts of alcohol (specifically ethanol) provided similar microbicidal performance to other compositions having a lower pH but which included increased amounts of ethanol. Thus, a reasonable degree of flexibility in formulating compositions of the invention is provided by judicious control of the pH and the amount of the lower alkyl monohydric alcohol present. Specific reference is made to the example formulations described hereinafter which demonstrate this effect. The pH of the inventive compositions is at least 5, but is preferably greater and in certain particularly preferred embodiments is substantially alkaline. While the pH of the composition may be 5 or greater, preferably the pH of the compositions is at least about 6, and more preferably is in the range of from about 7-14, especially in the range of about 9-12. Thus in preferred embodiments the pH of the treatment compositions (and/or microbicidal control system) is at least 5, and in order of increasing preference is at least 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5. In preferred embodiments, and in order of increasing preference the pH of the treatment compositions (and/or microbicidal control system) is not in excess of: 12.5, 12.4, 12.3, 12.2, 12.1, 12, 11.9, 11.8, 11.7, 11.6, 11.5, 11.4, 11.3, 11.2, 11.1, 11, 10.9, 10.8, 10.7, 10.6, 10.5, 10.4, 10.3, 10.2, 10.1, 10, 9.9, 9.8, 9.7, 9.6, 9.5. It is expected that compositions of the invention may have lower pHs, in the range of 1-14 if desired; however preferred pHs are indicated in the foregoing ranges and are demonstrated by the Examples. The pH of the surface treatment compositions may be established, adjusted and/or maintained by the addition of an effective amount of a pH adjustment constituent.

Optionally but preferably the treatment compositions of the invention include a pH adjusting constituent which may be used to establish and/or maintain, viz., buffer, a treatment composition at a desired pH or within a bounded pH range. Essentially any material which may increase or decrease the pH of the treatment composition is suitable as a pH adjusting constituent. Suitable pH adjusting constituents are one or more acids and/or bases whether such be based on organic and/or inorganic compounds or materials. By way of non-limiting example, pH adjusting agents include phosphorus containing compounds, monovalent and polyvalent salts such as of silicates, carbonates, and borates, certain acids and bases, tartrates and certain acetates. Further exemplary pH adjusting agents include mineral acids, basic compositions, and organic acids, which are typically required in only minor amounts. By way of further non-limiting example, pH buffering compositions include the alkali metal phosphates, polyphosphates, pyrophosphates, triphosphates, tetraphosphates, silicates, metasilicates, polysilicates, carbonates, hydroxides, and mixtures of the same. Certain salts, such as the alkaline earth phosphates, carbonates, and hydroxides, can also function as buffers. It may also be suitable to use as buffers such materials as aluminosilicates (zeolites), borates, aluminates and certain organic materials such as gluconates, succinates, maleates, citrates, and their alkali metal salts. Particularly useful and preferred is citric acid and metal salts thereof such as sodium citrate which are widely available and which are effective in providing these pH adjustment and buffering effects. Further exemplary and useful pH adjusting constituents include monoalkanolamines, dialkanolamines, trialkanolamines, and alkylalkanolamines such as alkyl-dialkanolamines, and dialkyl-monoalkanolamines. Such may also function as detersive surfactants. The alkanol and alkyl groups are generally short to medium chain length, that is, from 1 to 7 carbons in length. For di- and trialkanolamines and dialkyl-monoalkanolamines, these groups can be combined on the same amine to produce for example, methylethylhydroxypropylhydroxylamine. One of ordinary skill in the art can readily ascertain other members of this group. Preferred alkanolamines include monoethanolamine.

When present, the one or more pH adjusting constituents are included in amounts which are effective in establishing and/or maintaining the pH of a treatment composition at the desired pH value or within a range of pH values. Advantageously the one or more pH adjusting constituents comprise from about 0.001-2.5% wt., preferably from about 0.01-1.5% wt. of the treatment composition of which the one or more pH adjusting constituents form a part. Preferred pH adjusting constituents include those demonstrated in or more of the Examples. In certain preferred embodiments, one or more pH adjusting constituents are necessarily present and are to be understood as essential constituents of the treatment compositions.

The liquid inanimate surface treatment compositions of the invention may include one or more further optional constituents or materials which impart a desired technical and/or aesthetic features of the inventive compositions.

Although the compositions of the invention are largely aqueous in certain embodiments one or more organic solvents may be present. , particularly solvents other than one or more C₁-C₄ aliphatic alcohols and especially ethanol. Such are differentiated from the essential alkyl aliphatic monohydric alcohol constituent. Such further optional organic solvents may include one or more of: alcohols other than the essential lower alkyl aliphatic monohydric alcohol described previously, glycols, acetates, ether acetates, glycerols, as well as polyethylene glycols and glycol ethers. Mixtures of these further optional organic solvents can also be used. Typically such further one or more organic solvents are ones which have no appreciable microbicidal effect and are thus differentiated from the essential alkyl aliphatic monohydric alcohol constituent. Non-limiting examples of useful glycol ethers and examples include those glycol ethers having the general structure Rₐ—O—[CH₂—CH(R)—(CH₂)—O]—H, wherein Rₐ is C₁₋₂₀ alkyl or alkenyl, or a cyclic alkane group of at least 6 carbon atoms, which may be fully or partially unsaturated or aromatic; n is an integer from 1 to 10, preferably from 1 to 5; each R is selected from H or CH₃; and a is the integer 0 or 1. Specific and preferred solvents are selected from propylene glycol methyl ether, dipropylene glycol methyl ether, tripropylene glycol methyl ether, propylene glycol n-propyl ether, ethylene glycol n-butyl ether, diethylene glycol n-butyl ether, diethylene glycol methyl ether, propylene glycol, ethylene glycol, diethylene glycol monoethyl ether acetate and the like. When present such further optional one or more organic solvents may be present in any effective amount, preferably in amounts of between about 0.001-10% wt., and preferably between about 0.01-5% wt. based on the total weight of the treatment composition of which they form a part.

Optionally but in many instances preferably, the compositions of the invention include one or more surfactants, when such are not already present as an essential constituent. Examples of the major surfactant types that can be used to carry out the present invention include those already described previously, as well as one or more of: alkanolamines, alkylbenzenes, amine acetates, amine oxides, amines, sulfonated amines and amides, betaine derivatives, block polymers, carboxylated alcohol or alkylphenol ethoxylates, carboxylic acids and fatty acids, diphenyl sulfonate derivatives, ethoxylated alcohols, ethoxylated alkylphenols, ethoxylated amines and/or amides, ethoxylated fatty acids, ethoxylated fatty esters and oils, fatty esters, fluorocarbon-based surfactants, glycerol esters, glycol esters, heterocyclic-type products, imidazolines and imidazoline derivatives, isethionates, lanolin-based derivatives, lecithin and lecithin derivatives, lignin and lignin deriviatives, maleic or succinic anhydrides, methyl esters, monoglycerides and derivatives, olefin sulfonates, phosphate esters, phosphorous organic derivatives, polymeric (polysaccharides, acrylic acid, and acrylamide) surfactants, propoxylated and ethoxylated fatty acid alcohols or alkyl phenols, protein-based surfactants, quaternary surfactants other than those which exhibit a germicidal effect, sarcosine derivatives, silicone-based surfactants, soaps, sorbitan derivatives, sucrose and glucose esters and derivatives, sulfates and sulfonates of oils and fatty acids, sulfates and sulfonates, ethoxylated alkylphenols, sulfates of alcohols, sulfates of ethoxylated alcohols, sulfates of fatty esters, sulfonates of benzene, cumene, toluene and xylene, sulfonates of condensed naphthalenes, sulfonates of dodecyl and tridecylbenzenes, sulfonates of naphthalene and alkyl naphthalene, sulfonates of petroleum, sulfosuccinamates, sulfosuccinates and derivatives, taurates, thio and mercapto derivatives, tridecyl and dodecyl benzene sulfonic acids, as well as further surfactants known to the art but not elucidated here.

Typically however the use of anionic surfactants is to be avoided as such would be expected to form insoluble complexes quaternary ammonium compound which provides a germicidal or microbicidal benefit is concurrently present.

The treatment compositions of the invention may optionally include one or more acids, which include not only organic and inorganic acids but also acid salts of organic acids. Preferred examples of the organic acid to be used in the present invention include linear aliphatic acids such as formic acid, acetic acid, propionic acid, butyric acid and valeric acid; dicarboxylic acids such as oxalic acid, malonic acid, succinic acid, glutaric acid, adipic acid, pimelic acid, fumaric acid and maleic acid; acidic amino acids such as glutamic acid and aspartic acid; and hydroxy acids such as glycolic acid, lactic acid, hydroxyacrylic acid, alpha-hydroxybutyric acid, glyceric acid, tartronic acid, malic acid, tartaric acid and citric acid, as well as acid salts of these organic acids. Exemplary inorganic acids include phosphoric acid, potassium dihydrogenphosphate, sodium dihydrogenphosphate, sodium sulfite, potassium sulfite, sodium pyrosulfite (sodium metabisulfite), potassium pyrosulfite (potassium metabisulfite), acid sodium hexametaphosphate, acid potassium hexametaphosphate, acid sodium pyrophosphate, acid potassium pyrophosphate and sulfamic acid. These acids can be used singly or as a mixture of two or more inorganic and/or organic acids. Such one or more acids may be used to adjust the pH of the inventive compositions, and/or buffer the pH of the treatment compositions. When present, these may be included in effective amounts. Particularly useful is citric acid and metal salts thereof such as sodium citrate which are widely available and which are effective in providing these pH adjustment and buffering effects. These should be screened however to ensure that they do not undesirably complex with or in other ways deactivate any quaternary ammonium compound(s) which may be present.

The treatment compositions of the invention may also include one or more further compounds, constituents or materials which provide an ancillary microbicidal benefit or effect. These are distinguished from the essential constituents of the invention described above. When present, they may be included in amounts which are effective in order to provide an ancillary microbicidal benefit. Non-limiting examples of such materials include non-cationic microbicidal agents which are particularly useful in the present invention: pyrithiones (especially zinc pyrithione which is also known as ZPT), dimethyldimethylol hydantoin (Glydant), methylchloroisothiazolinone/methylisothiazolinone (Kathon C G), sodium sulfite, sodium bisulfite, imidazolidinyl urea (Germall 115), diazolidinyl urea (Germaill II), benzyl alcohol, 2-bromo-2-nitropropane-1,3-diol (Bronopol), formalin (formaldehyde), iodopropenyl butylcarbamate (Polyphase P100), chloroacetamide, methanamine, methyldibromonitrile glutaronitrile (1,2-Dibromo-2,4-dicyanobutane or Tektamer), glutaraldehyde, 5-bromo-5-nitro-1,3-dioxane (Bronidox), phenethyl alcohol, o-phenylphenol/sodium o-phenylphenol, sodium hydroxymethylglycinate (Suttocide A), polymethoxy bicyclic oxazolidine (Nuosept C), dimethoxane, thimersal dichlorobenzyl alcohol, captan, chlorphenenesin, dichlorophene, chlorbutanol, glyceryl laurate, halogenated diphenyl ethers like 2,4,4-trichloro-2-hydroxy-diphenyl ether (Triclosan or TCS), 2,2-dihydroxy-5,5-dibromo-diphenyl ether, phenolic compounds like phenol, 2-methyl phenol, 3-methyl phenol, 4-methyl phenol, 4-ethyl phenol, 2,4-dimethyl phenol, 2,5-dimethyl phenol, 3,4-dimethyl phenol, 2,6-dimethyl phenol, 4-n-propyl phenol, 4-n-butyl phenol, 4-n-amyl phenol, 4-tert-amyl phenol, 4-n-hexyl phenol, 4-n-heptyl phenol, mono- and poly-alkyl and aromatic halophenols such as p-chlorophenol, methyl p-chlorophenol, ethyl p-chlorophenol, n-propyl p-chlorophenol, n-butyl p-chlorophenol, n-amyl p-chlorophenol, sec-amyl p-chlorophenol, n-hexyl p-chlorophenol, cyclohexyl p-chlorophenol, n-heptyl p-chlorophenol, n-octyl p-chlorophenol, o-chlorophenol, methyl o-chlorophenol, ethyl o-chlorophenol, n-propyl o-chlorophenol, n-butyl o-chlorophenol, n-amyl o-chlorophenol, tert-amyl o-chlorophenol, n-hexyl o-chlorophenol, n-heptyl o-chlorophenol, o-benzyl p-chlorophenol, o-benzyl-m-methyl p-chlorophenol, o-benzyl-m, m-dimethyl p-chlorophenol, o-phenylethyl p-chlorophenol, o-phenylethyl-m-methyl p-chlorophenol, 3-methyl p-chlorophenol, 3,5-dimethyl p-chlorophenol, 6-ethyl-3-methyl p-chlorophenol, 6-n-propyl-3-methyl p-chlorophenol, 6-iso-propyl-3-methyl p-chlorophenol, 2-ethyl-3,5-dimethyl p-chlorophenol, 6-sec-butyl-3-methyl p-chlorophenol, 2-iso-propyl-3,5-dimethyl p-chlorophenol, 6- diethylmethyl-3-methyl p-chlorophenol, 6-iso-propyl-2-ethyl-3-methyl p-chlorophenol, 2-sec-amyl-3,5-dimethyl p-chlorophenol 2-diethylmethyl-3,5-dimethyl p-chlorophenol, 6-sec-octyl-3-methyl p-chlorophenol, p-chloro-m-cresol, p-bromophenol, methyl p-bromophenol, ethyl p-bromophenol, n-propyl p-bromophenol, n-butyl p-bromophenol, n-amyl p-bromophenol, sec-amyl p-bromophenol, n-hexyl p-bromophenol, cyclohexyl p-bromophenol, o-bromophenol, tert-amyl o-bromophenol, n-hexyl o-bromophenol, n-propyl-m,m-dimethyl o-bromophenol, 2-phenyl phenol, 4-chloro-2-methyl phenol, 4-chloro-3-methyl phenol, 4-chloro-3,5-dimethyl phenol, 2,4-dichloro-3,5-dimethylphenol, 3,4,5,6-terabromo-2-methylphenol, 5-methyl-2-pentylphenol, 4-isopropyl-3-methylphenol, para-chloro-meta-xylenol, dichloro meta xylenol, chlorothymol, 5-chloro-2-hydroxydiphenylmethane, resorcinol and its derivatives including methyl resorcinol, ethyl resorcinol, n-propyl resorcinol, n-butyl resorcinol, n-amyl resorcinol, n-hexyl resorcinol, n-heptyl resorcinol, n-octyl resorcinol, n-nonyl resorcinol, phenyl resorcinol, benzyl resorcinol, phenylethyl resorcinol, phenylpropyl resorcinol, p-chlorobenzyl resorcinol, 5-chloro 2,4-dihydroxydiphenyl methane, 4-chloro 2,4-dihydroxydiphenyl methane, 5-bromo 2,4-dihydroxydiphenyl methane, and 4-bromo 2,4-dihydroxydiphenyl methane, bisphenolic compounds like 2,2-methylene bis (4-chlorophenol), 2,2-methylene bis (3,4,6-trichlorophenol), 2,2-methylene bis (4-chloro-6-bromophenol), bis (2-hydroxy-3,5-dichlorophenyl) sulphide, and bis (2-hydroxy-5-chlorobenzyl)sulphide, benzoic esters (parabens) like methylparaben, propylparaben, butylparaben, ethylparaben, isopropylparaben, isobutylparaben, benzylparaben, sodium methylparaben, and sodium propylparaben, halogenated carbanilides (e.g., 3,4,4-trichlorocarbanilides (Triclocarban or TCC), 3-trifluoromethyl-4,4-dichlorocarbanilide, 3,3,4-trichlorocarbanilide, etc.).

Of these, preferred are phenol based non-cationic microbicidals, especially those based on one or more phenolic compounds, particularly 2-hydroxydiphenyl compounds which may be exemplified by the following classes of compounds:

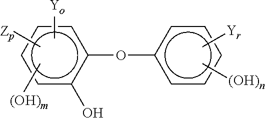

wherein Y is chlorine or bromine, Z is SO₂ H, NO₂, or C₁-C₄ alkyl, r is 0 to 3, o is 0 to 3, p is 0 or 1, m is 0 or 1, and n is 0 or 1. In preferred embodiments, Y is chlorine or bromine, m is 0, n is 0 or 1, o is 1 or 2, r is 1 or 2, and p is 0, and according to especially preferred embodiments, Y is chlorine, m is 0, n is 0, o is 1, r is 2, and p is 0.

Particularly useful 2-hydroxydiphenyl compounds include those which may be represented by the structure:

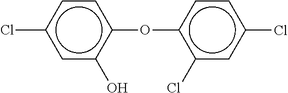

which is commonly referred to as “TRICLOSAN” and which is presently commercially available from Ciba Specialty Chemicals Corp., as well as halogenated carbanilides, e.g., TCC.

Further exemplary useful phenolic based disinfecting agents include 2,2′-hydroxy-5,5′-dibromo-diphenyl ether which may be represented by the structure:

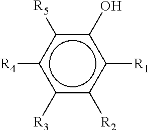

wherein R₁ is hydro, hydroxy, C₁-C₄ alkyl, chloro, nitro, phenyl, or benzyl; R₂ is hydro, hydroxy, C₁-C₆ alkyl, or halo; R₃ is hydro, C₁-C₆ alkyl, hydroxy, chloro, nitro, or a sulfur in the form of an alkali metal salt or ammonium salt; R₄ is hydro or methyl, and R₅ is hydro or nitro. Halo is bromo or, preferably, chloro.

Specific examples of phenol derivatives include, but are not limited to, chlorophenols (o-, m-, p-), 2,4-dichlorophenol, p-nitrophenol, picric acid, xylenol, p-chloro-m-xylenol, cresols (o-, m-, p-), p-chloro-m-cresol, pyrocatechol, resorcinol, 4-n-hexylresorcinol, pyrogallol, phloroglucin, carvacrol, thymol, p-chlorothymol, o-phenylphenol, o-benzylphenol, p-chloro-o-benzylphenol, phenol, 4-ethylphenol, and 4-phenolsulfonic acid.

Still further useful phenol derivatives include those which may be represented by the structure:

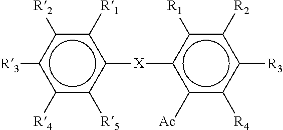

wherein X is sulfur or a methylene group, R₁ and R′₁ are hydroxy, and R₂, R′₂, R₃, R′₃, R₄, R′₄, R₅, and R′₅, independent of one another, are hydro or halo. Specific, nonlimiting examples of diphenyl compounds are hexachlorophene, tetrachlorophene, dichlorophene, 2,3-dihydroxy-5,5′-dichlorodiphenyl sulfide, 2,2′-dihydroxy-3,3′, 5,5′-tetrachlorodiphenyl sulfide, 2,2′-dihydroxy-3,5′5,5′, 6,6′-hexachlorodiphenyl sulfide, and 3,3′-dibromo-5,5′-dichloro-2,2′-dihydroxydiphenylamine. Of the foregoing, a particularly useful phenol derivative is commonly referred to as triclocarban, or 3,4,4′-trichlorocarbanilide as well as derivatives thereto. When present, one or more such further compounds, constituents or materials which provide an ancillary microbicidal benefit or effect may be present in effective amounts, e.g., in amounts of up to about 5% wt., although depending upon the efficacy of one or more selected such further compounds, constituents or materials are usually effective in reduced amounts, e.g., 0.001-2% wt. of the treatment composition.

The treatment compositions of the invention may optionally include a fragrance constituent, which may be based on natural and/or synthetic fragrances and most commonly are mixtures or blends of a plurality of such fragrances, optionally in conjunction with a carrier such as an organic solvent or a mixture of organic solvents in which the fragrances are dissolved, suspended or dispersed. Such may be natural fragrances, e.g, natural extracts of plants, fruits, roots, stems, leaves, wood extracts, e.g. terpineols, resins, balsams, animal raw materials, e.g., civet and beaver, as well as typical synthetic perfume compounds which are frequently products of the ester, ether, aldehyde, ketone, alcohol and hydrocarbon type, e.g., benzyl acetate, linalyl acetate, citral, citronellal, methyl cedryl ketone, eugenol, isoeugenol, geraniol, linalool, and Typically it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, labolanum oil and lavendin oil. When present in a treatment composition, in accordance with certain of the preferred embodiments, the fragrance constituent may be present in any effective amount such that it can be discerned by a consumer of the composition, however such is advantageously present in amounts of up to about 1% wt., preferably are present in amounts of from about 0.00001% wt. to about 0.5% wt., and most preferably is present in an amount of from about 0.0001% wt. to 0.5% wt. based on the total weight of the treatment composition of which it forms a part.

A further optional constituent of the treatment compositions of the invention include colorant, such as dyes and pigments which may be used to impart a color to the compositions of which they form a part.

The treatment compositions of the invention may also optionally include a preservative constituent which is used to control undesired microorganisms within the treatment composition particularly when the treatment composition is in long-term storage and at elevated temperatures. While these are normally not present due to the microbicidal efficacy of the compositions as taught herein, such ancillary preservative constituents may be included in minor but effective amounts. Nonlimiting examples include one or more of parabens, including methyl parabens and ethyl parabens, glutaraldehyde, formaldehyde, 2-bromo-2-nitropropoane-1,3-diol, 5-chloro-2-methyl-4-isothiazolin-3-one, 2-methyl-4-isothiazoline-3-one, and mixtures thereof. One exemplary composition is a combination 5-chloro-2-methyl-4-isothiazolin-3-one and 2-methyl-4-isothiazolin-3-one where the amount of either component may be present in the mixture anywhere from 0.001 to 99.99 weight percent, based on the total amount of the preservative. Further exemplary useful preservatives include those which are commercially including a mixture of 5-chloro-2-methyl-4-isothiazolin-3-one and 2-methyl-4-isothiazolin-3-one marketed under the trademark KATHON® CG/ICP as a preservative composition presently commercially available from Rohm and Haas (Philadelphia, Pa.).

A further optional constituent in the inventive treatment compositions is one or more chelating agents. Exemplary useful chelating agents include those known to the art, including by way of non-limiting example; aminopolycarboxylic acids and salts thereof wherein the amino nitrogen has attached thereto two or more substituent groups. Preferred chelating agents include acids and salts, especially the sodium and potassium salts of ethylenediaminetetraacetic acid, diethylenetriaminepentaacetic acid, N-hydroxyethylethylenediaminetriacetic acid, and of which the sodium salts of ethylenediaminetetraacetic acid may be particularly advantageously used. Such chelating agents may be omitted, or they may be included in generally minor amounts such as from about 0.001-0.5% wt. based on the weight of the chelating agents and/or salt forms thereof. When present, advantageously, such chelating agents are included in the present inventive composition in amounts from about 0.001-5% wt., but are most desirably present in reduced weight percentages from about 0.01-0.5% wt.

The amounts or presence of chelating agents should be carefully controlled and may in some preferred embodiments are excluded from the treatment compositions. This is due the fact that the presence of chelating agents may undesirably form insoluble complexes with the Cu(I) and/or Cu(II) ions present in the compositions, and thus reduce or remove their beneficial effects in the treatment compositions. Desirably when such are present they are included in only limited amounts so as not to deleteriously affect the improved microbicidal efficacy of the inventive compositions, but in particularly preferred embodiments such chelating agents are desirably excluded from the compositions of the invention

When one or more such further optional constituents are present in the treatment compositions, preferably their cumulative amount does not exceed about 25% wt. and preferably does not exceed about 20% wt., of the treatment composition of which they form a part.

The inventive compositions are preferably liquids which have a viscosity in the range of about 200 centipoise (“cP”) or less, preferably and in order of increasing preference, viscosities of 150 cP, 100 cP, 75 cP, 50 cP, 25 cP, 20 cP, 15 cP, 10 cP, 5 cP, 3 cP, 2 cP, and 1 cP, when measured using conventional quantitative method, e.g., as measured at 20° C. or 25° C. by a Brookfield Type LVT or Type RVT viscometer using a standard spindle provided by that manufacturer and measuring the samples at room temperature (20-25° C.).

As the treatment compositions taught herein are used to treat inanimate surfaces including porous and nonporous surfaces and are not provided as a topical skin treatment composition or personal care composition or for that matter as a wound dressing or a preparation for use in wound dressings, the treatment compositions most preferably exclude (unless already described previously) as constituents known-art certain additives and adjuvants which are conventional in the cosmetic, pharmaceutical or dermatological field, specifically hydrophilic or lipophilic gelling agents, hydrophilic or lipophilic active agents, humectants, opacifiers, light stabilizers including UV absorbers, and Polyquaternium type polymers.

The treatment compositions most preferably exclude (unless already described previously) thickener components especially one or more of polysaccharide thickeners such as cellulose, alkyl celluloses, alkoxy celluloses, hydroxy alkyl celluloses, naturally occurring polysaccharide polymers such as xanthan gum, guar gum, locust bean gum, tragacanth gum, or derivatives thereof, polycarboxylate polymers, polyacrylamides, clays, and mixtures thereof.

The treatment compositions of the invention are not provided with an aerosol propellant gas or constituent, and are not packaged or sold as vendible articles in pressured containers, e.g., aerosol canisters. The surface treatment compositions are pourable and pumpable, and may be dispensed by pumping the composition through a manually operated or a power driven (e.g., motor driven, pressure driven) dispensing device, such as a sprayer, viz “trigger” sprayer or spray pump affixed to a container containing a quantity of the surface treatment composition. The surface treatment composition may also be a pourable composition which may be dispensed from the open end of a suitable flask, bottle or other container, or may be dispensed via a suitable nozzle or spout, e.g., which may be operated by either inversion of the container, and optionally compressing some or part of the container, so to expel it from the container to a surface to be treated. Between such dispensing operations; however, the contents of such a container which includes the surface treatment composition are not pressurized. Thus a further aspect of the invention provides a closed container containing the inventive composition as described herein.

The treatment compositions may also be dispensed, e.g. to a surface, or delivered to an airspace, by means of a mist generator means. Such a mist generator means typically includes an element or member which operates to comminute the unpressurized liquid treatment composition into small particles which form a mist, e.g. nebulize or atomize the unpressurized liquid treatment composition. Such a mist generator means may also be considered an aerosol delivery system which is however not generated from a device wherein the treatment composition also includes a propellant constituent. The mist generator means may comprise a vibrating member which includes a metal or ceramic plate; the plate may be solid or porous, or micropierced in the form of a grid or in the form of one or more segments or slots passing through the vibrating member, and a piezoelectric actuator which, when operated, causes vibratory motion in the vibrating member. Alternately, the mist generator means may be an electrostatic spray device. Alternately the mist generator means may be an ultrasonic nozzle device. Such devices are known to the art. Nonlimiting examples of such mist generators and devices which include such mist generator means include those disclosed in one or more of: U.S. Pat. No. 5,743,251, U.S. Pat. No. 6,234,167, U.S. Pat. No. 6,491,233, U.S. Pat. No. 6,501,052, U.S. Pat. No. 6,516,796, U.S. Pat. No. 6,568,390, U.S. Pat. No. 6,640,050, U.S. Pat. No. 6,681,998, U.S. Pat. No. 6,766,220, U.S. Pat. No. 6,772,757, U.S. Pat. No. 6,804,458, U.S. Pat. No. 6,883,516, U.S. Pat. No. 7,229,029, US 2007/0011940, US 2007/0169775, US 2007/0235555, US 2008/00419272, US 2009/0121043, US 2009/0272818, the entire contents of each of which are herein incorporated by reference thereto.

The mist generator means may be an ultrasonic nozzle device. Such ultrasonic nozzle devices may be obtained from commercial sources, e.g., Sono-Tek, Inc. (Milton, N.Y., USA) as well as Sonaer Inc., (Farmingdale, N.Y., USA) as well as being disclosed in published patent applications, US 2009/0254020, and US 2009/0224066, the contents of which are herein incorporated by reference.

The treatment compositions may also be dispensed, e.g. to a surface, or delivered to an airspace, by means of evaporation of the unpressurized liquid treatment composition particularly to an airspace. For example, the unpressurized liquid treatment composition may be provided in a container for containing the same, and a wick inserted into the unpressurized liquid treatment composition which wick also extends outwardly from the container and from whence the unpressurized liquid treatment composition may evaporate or be otherwise delivered to a surface or to an airspace. Nonlimiting examples of such devices include those disclosed in one or more of: U.S. Pat. No. 7,168,631, U.S. Pat. No. 6,699,432, U.S. Pat. No. 6,580,875, U.S. Pat. No. 4,898,328, the entire contents of each of which are herein incorporated by reference thereto.

When used to provide an air treatment benefit, it is preferred that the surface treatment composition comprise at least about 30% wt. of the lower alkyl aliphatic monohydric alcohol constituent, in addition to the further essential constituents heretofore described.

Thus a further aspect of the invention provides a closed container containing the inventive composition as described herein.

The treatment compositions of the invention may also be supplied within a water dispersible, water miscible or water soluble sachet or pouch or water-soluble package; such may be formed from a water soluble material, such as a water soluble or water dispersible polymeric film (e.g. polyvinyl alcohol), or alternately may be formed from a water insoluble material, such as a water insoluble polymeric film. Additionally the sachet, pouch or package may be formed in a manner where only part of the sachet is physically breachable or only part of the sachet, pouch or package is water soluble or dispersible. Thus a further aspect of the invention provides a closed, a water dispersible, a water miscible or a water soluble sachet or pouch containing the inventive composition as described herein.

The treatment compositions can also be applied to a hard surface by using a wet wipe. The wipe can be of a woven or non-woven nature. Fabric substrates can include nonwoven or woven pouches, sponges, in the form of abrasive or non-abrasive cleaning pads. Such fabrics are known commercially in this field and are often referred to as wipes. Such substrates can be resin bonded, hydroentanged, thermally bonded, meltblown, needlepunched or any combination of the former.

The nonwoven fabrics may be a combination of wood pulp fibers and textile length synthetic fibers formed by well known dry-form or wet-lay processes. Synthetic fibers such as rayon, nylon, orlon and polyester as well as blends thereof can be employed. The wood pulp fibers should comprise about 30 to about 60 percent by weight of the nonwoven fabric, preferably about 55 to about 60 percent by weight, the remainder being synthetic fibers. The wood pulp fibers provide for absorbency, abrasion and soil retention whereas the synthetic fibers provide for substrate strength and resiliency.

The substrate of the wipe may also be a film forming material such as a water soluble polymer. Such self-supporting film substrates may be sandwiched between layers of fabric substrates and heat sealed to form a useful substrate. The free standing films can be extruded utilizing standard equipment to devolatilize the blend. Casting technology can be used to form and dry films, or a liquid blend can be saturated into a carrier and then dried in a variety of known methods.

The treatment compositions of the present invention are absorbed onto the wipe to form a saturated wipe and sold as a vendible product. The wipe can then be sealed individually in a pouch which can then be opened when needed or a multitude of wipes can be placed in a container for use on an as-needed basis. The container, when closed, is sufficiently sealed to prevent evaporation of any components from the compositions. Thus a further aspect of the invention provides a closed container containing one or more wipes which include the treatment composition as described herein.

The treatment compositions of the invention may be used to provide or impart a microbicidal effect on treated inanimate surfaces. Preferably the surface treatment compositions are characterized in exhibiting a microbicidal benefit when tested against one or more challenge microorganisms according to one or more of the following standardized test protocols: ASTM E1052-96(2002) Standard Test Method for Efficacy of Antimicrobial Agents against Viruses in Suspension, or ASTM E1053-11 Standard Test Method to Assess Virucidal Activity of Chemicals Intended for Disinfection of Inanimate, Nonporous Environmental Surfaces, or

European Standard Surface Test, EN13697 or AOAC Germicidal Spray Products as Disinfectant Test Method, AOAC Index, 17^{th} Ed. (2000) against one or more challenge microorganisms. In particularly preferred embodiments the treatment compositions exhibit a high degree of microbicidal efficacy against various undesirable microorganisms (sometimes referred to as ‘pathogens’) including various bacteria, viruses, and fungi. In particularly preferred embodiments treatment compositions of the invention exhibit a high degree of microbicidal efficacy against poliovirus type 1 (Sabin) (“PV1”).

The surface treatment compositions may be applied to inanimate surfaces in order to impart a cleaning effect thereto, but preferably are applied to impart a microbicidal benefit thereto. Inanimate surfaces include hard surfaces, which are typically nonporous hard surfaces. By way of example, hard surfaces include surfaces composed of refractory materials such as: glazed and unglazed tile, brick, porcelain, ceramics as well as stone including marble, granite, and other stones surfaces; glass; metals; plastics e.g. polyester, vinyl; fiberglass, Formica®, Coriant and other hard surfaces known to the industry. Hard surfaces which are to be particularly denoted are lavatory fixtures, lavatory appliances (toilets, bidets, shower stalls, bathtubs and bathing appliances), wall and flooring surfaces especially those which include refractory materials and the like. Further hard surfaces which are particularly denoted are those associated with kitchen environments and other environments associated with food preparation. Hard surfaces which are those associated with hospital environments, medical laboratories and medical treatment environments. Inanimate surfaces which may be treated by the surface treatment compositions of the invention include soft surfaces, non-limiting examples of which include: carpets, rugs, upholstery, curtains and drapes, fabrics, textiles, garments, and the like

The treatment compositions described herein may also be used to provide an air treatment benefit if they are sprayed or dispersed into the air, particularly if the surface treatment composition is provided as comminuted particles, viz., droplets within an airspace, such that the treatment composition contacts said airspace and provides a technical benefit thereto, e.g., fragrancing, odor masking, odor elimination, malodour neutralization, air sanitization, and the like. The method may be practiced within an open airspace, e.g., a larger volume such as a room, public space within the interior of a building, a cabin or compartment within a vehicle, as well as within a closed container or other relatively smaller space, e.g., the interior of a storage cabinet, a closet, a shower stall, a garbage container or refuse bin, and the like. When used to provide an air treatment benefit, it is preferred that the surface treatment composition comprise at least about 30% wt. of the lower alkyl aliphatic monohydric alcohol constituent, in addition to the further essential constituents heretofore described.

The treatment compositions may also be dispensed, e.g. to a surface, or delivered to an airspace, by means of a mist generator means. Such a mist generator means typically includes an element or member which operates to comminute the unpressurized liquid treatment composition into small particles which form a mist, e.g. nebulize or atomize the unpressurized liquid treatment composition. Such a mist generator means may also be considered an aerosol delivery system which is however not generated from a device wherein the treatment composition also includes a propellant constituent. The mist generator means may comprise a vibrating member which includes a metal or ceramic plate; the plate may be solid or porous, or micropierced in the form of a grid or in the form of one or more segments or slots passing through the vibrating member, and a piezoelectric actuator which, when operated, causes vibratory motion in the vibrating member. Alternately, the mist generator means may be an electrostatic spray device. Alternately the mist generator means may be an ultrasonic nozzle device. Such devices are known to the art. Nonlimiting examples of such mist generators and devices which include such mist generator means include those disclosed in one or more of: U.S. Pat. No. 5,743,251, U.S. Pat. No. 6,234,167, U.S. Pat. No. 6,491,233, U.S. Pat. No. 6,501,052, U.S. Pat. No. 6,516,796, U.S. Pat. No. 6,568,390, U.S. Pat. No. 6,640,050, U.S. Pat. No. 6,681,998, U.S. Pat. No. 6,766,220, U.S. Pat. No. 6,772,757, U.S. Pat. No. 6,804,458, U.S. Pat. No. 6,883,516, U.S. Pat. No. 7,229,029, US 2007/0011940, US 2007/0169775, US 2007/0235555, US 2008/00419272, US 2009/0121043, US 2009/0272818, the entire contents of each of which are herein incorporated by reference thereto.

The mist generator means may be an ultrasonic nozzle device. Such ultrasonic nozzle devices may be obtained from commercial sources, e.g., Sono-Tek, Inc. (Milton, NY, USA) as well as Sonaer Inc., (Farmingdale, N.Y., USA) as well as being disclosed in published patent applications, US 2009/0254020, and US 2009/0224066, the contents of which are herein incorporated by reference.

The treatment compositions may also be dispensed, e.g. to a surface, or delivered to an airspace, by means of evaporation of the unpressurized liquid treatment composition particularly to an airspace. For example, the unpressurized liquid treatment composition may be provided in a container for containing the same, and a wick inserted into the unpressurized liquid treatment composition which wick also extends outwardly from the container and from whence the unpressurized liquid treatment composition may evaporate or be otherwise delivered to a surface or to an airspace. Nonlimiting examples of such devices include those disclosed in one or more of: U.S. Pat. No. 7,168,631, U.S. Pat. No. 6,699,432, U.S. Pat. No. 6,580,875, U.S. Pat. No. 4,898,328, the entire contents of each of which are herein incorporated by reference thereto.

Thus a further aspect of the invention provides a closed container containing the inventive composition as described herein.

As certain embodiments of the invention there are provided processes for the treatment of surfaces, or air, including inanimate hard surfaces and inanimate soft surfaces which method includes the step of: contacting such a surface which is in need of treatment or upon which the presence of one or more undesirable microorganisms are suspected or are known to be present, with an effective amount of a surface treatment composition as described herein to provide a surface treatment benefit thereto, preferably to provide a microbicidal benefit to the surface, particularly against various undesirable microorganisms (sometimes referred to as ‘pathogens’) including various bacteria, mycobacteria, viruses, and fungi, and particularly preferably against poliovirus type 1 (Sabin) (“PV1”). Such methods require the application of an effective amount of a treatment composition as taught herein to such surfaces, so that the desired microbicidal benefit is imparted to the treated surface. Desirably such an effective amount is a sufficient amount of a treatment composition which will provide at least a 1 log₁₀ reduction, more preferably at least, and in order of increasing preference, a 1.25, 1.5, 1.75, 2, 2.25, 2.5, 2.75, 3, 3.25, 3.5, 3.75, 4, 4.25, 4.5, 4.75, and 5 logᵢₙ reduction against poliovirus type 1 (Sabin) (“PV1”) in accordance with one or more of the testing protocols described hereinafter, and/or degrees of microbicidal efficacy of poliovius type 1 (Sabin) or other challenge microorganism, as are demonstrated with reference to one or more of the Examples.

With reference to the Examples described later, and with reference to Tables C and 1, the disclosed compositions were subjected to one or more of the following test protocols in order to evaluate their microbiocidal efficacy against one or more of the other challenge microorganisms which are identified on Table B. As is known in the art, amongst the most difficult to control or eradicate are non-enveloped viruses, such as poliovirus type 1 (Sabin,) and while microbicidal efficacy against the poliovirus type 1 (Sabin) presumptively demonstrates that the same composition would be expected to be effective against the bacteria and the other non-enveloped viruses disclosed on Table B, however, the converse is not expected to be true by a skilled artisan. Thus, for example, while a composition which exhibits good microbicidal efficacy against a Gram-positive or Gram-negative bacteria, such would not be expected to be particularly effective against the poliovirus type 1 (Sabin), while the converse would be expected to be true. Further, even demonstrated efficacy of a composition against a relatively easier to control or eradicate non-enveloped viruses, such as the rotaviruses, or human adenovirus would not necessarily be expected by a skilled artisan to be particularly effective against the poliovirus type 1 (Sabin), while the converse would be expected to be true.

Preferred and particularly preferred treatment compositions of the invention demonstrate a microbicidal benefit when tested according to the standardized protocol outlined in ASTM E1052-96(2002) Standard Test Method for Efficacy of Antimicrobial Agents against Viruses in Suspension. This test is per se, known to the art. Preferred treatment compositions exhibit a microbicidal benefit against one, but preferably against two or more of the following challenge organisms, specifically “challenge” non-enveloped viruses: hepatitis A (supplied as hepatitis A virus, strain MH-175 ex. Dr. Mark Sobsey, University of North Carolina, Chapel Hill, N.C.; human adenovirus type 5 (supplied as ATCC VR-5); feline calicivirus strain F-9 (supplied as ATCC VR-782); herpes simplex type 1 (supplied as ATCC VR-1493); human rhinovirus type 14 strain 1059 (supplied as ATCC VR-284), and especially preferably against poliovirus type 1 (Sabin) (supplied by U.S. Centers for Disease Control and Prevention (CDC)), in accordance with this test protocol. As is known to the skilled artisan, of these forgoing challenge viruses, the most resistant to control or eradicate is the poliovirus type 1 (Sabin) and is it commonly presumed that any composition which shows an effective degree of control or eradication against the poliovirus type 1 (Sabin) virus will exhibit an even greater degree of control or eradication of the further prior listed viruses. As is known from the literature, e.g.,. Hierarchy of susceptibility of viruses to environmental surface disinfectants: a predictor of activity against new and emerging viral pathogens. J. AOAC International 90:1655-1658, Sattar, S. A. (2007) the efficacy of a composition in controlling or eradicating poliovirus type 1 (Sabin) provides an excellent prediction of the composition's efficacy against further challenge non-enveloped viruses.

Preferred treatment compositions of the invention demonstrate a microbicidal benefit when tested according to the standardized protocol outlined in ASTM E1053-11 Standard Test Method to Assess Virucidal Activity of Chemicals Intended for Disinfection of Inanimate, Nonporous Environmental Surfaces. This test is also, per se, known to the art. Preferred treatment compositions exhibit a microbicidal benefit against poliovirus type 1 (Sabin) (supplied by U.S. Centers for Disease Control and Prevention (CDC)), in accordance with this test protocol. Again, as is known to the skilled artisan, of these forgoing challenge viruses the most resistant to control or eradication is the poliovirus type 1 (Sabin) and is it commonly presumed that any composition which shows an effective degree of control or eradication against the poliovirus type 1 (Sabin) will exhibit an even greater degree of control or eradication of many other viruses, including hepatitis A virus, feline calicivirus strain F-9, Herpes simplex type 1 and human rhinovirus type 14 strain 1059 as identified above.

Preferred treatment compositions of the invention demonstrate a microbicidal benefit when tested according to the standardized protocol outlined in European Standard Surface Test, EN13697. This test too is, per se, known to the art. Preferred treatment compositions exhibit a microbicidal benefit against one or more of the following bacteria or fungi: Staphylococcus aureus(supplied as ATCC 6538); Escherichia coli (supplied as ATCC 10536); Pseudomonas aeruginosa (supplied as ATCC 15442); Enterococcus hirae (supplied as ATCC 10541) and/or the fungus, Aspergillus niger (supplied as ATCC 16404) and/or the fungus Trichophyton menhigrophytes (supplied as ATCC 9533) in accordance with the protocols of the test.

Certain preferred treatment compositions of the invention also demonstrate a microbicidal benefit when tested according to the standardized protocol identified as the AOAC Germicidal Spray Products as Disinfectant Test Method, AOAC Index, 17^{th} Ed. (2000). This test is also, per se, known to the art. Preferred treatment compositions exhibit a microbicidal benefit against one or more of the following bacteria or fungi: Staphylococcus aureus (“S. aureus” or “Sa”) (supplied as ATCC 6538); Escherichia coli (“E. coli”, or “Ec”) (supplied as ATCC 10536), in accordance with the protocols of the test.

The following examples below illustrate exemplary formulations as well as preferred embodiments of the invention. It is to be understood that these examples are provided by way of illustration only and that further useful formulations falling within the scope of the present invention and the claims may be readily produced by one of ordinary skill in the art without deviating from the scope and spirit of the invention.

### EXAMPLES

A number of treatment compositions as well as a number of comparative compositions were produced and are described on the following Tables. In these identified compositions, the constituents were used “as supplied” from their respective suppliers and may constitute less than 100% wt. “actives”, or may have been supplied as constituting 100% wt. “active” of the named compound, as indicated below. Treatment compositions which are considered to fall within the scope of the present invention are identified by a digit prepended with the letter “E” which indicates this to be an “example” composition, while compositions provided only for the purposes of comparison are identified by a digit prepended with the letter “C”, which indicates this to be a comparative composition and falling outside of the scope of the present invention. In certain of the treatment compositions, one or more constituents, e.g, a pH adjusting agent, or deionized water was added in “quantum sufficient” “q.s.” in order to provide a desired pH or to provide a sufficient mass in order to provide 100% wt. of each composition. The example compositions disclosed hereinafter include certain presently preferred embodiments of the invention. The comparative compositions are presented on Table C, while treatment compositions of the invention are identified on one or more of the further tables, e.g, Table 1.

The compositions disclosed on the following tables were produced by simple mixing, under stirring, of the identified constituents, generally in accordance with the following protocol. To a suitably sized laboratory beaker outfitted with a mechanical stirrer or a magnetic stirrer, was first supplied a major proportion of the deionized water. All of the constituents, as well as the laboratory beaker were at room temperature (approx. 20° C.) and as the beaker was open, mixing was at normal atmospheric pressure. Thereafter under stirring conditions (approx. 300 rpm) was added the source of copper ions, and mixing continued until this material was dissolved. Stirring continued for a further 15-30 minutes to ensure a homogenous mixture, to which was next added the alcohol constituent. Subsequently were added any remaining constituents including any further quantity of pH adjusting constituents (if present) in order to establish the desired pH of the surface treatment composition. Constituents identified as being added “q.s.” were added in order to adjust the pH of the formed composition or to bring the weight of the formed composition to 100% wt. Stirring continued for a further 1-15 minutes to ensure the formation of a homogenous mixture, after which the surface treatment composition was withdrawn from the beaker and used or tested.

These compositions as identified below were formed using the constituents identified on the following Table A which identifies the specific constituents used.

TABLE A

| | Constituents |
|---|---|
| CuSO4•5H2O | CuSO₄•5H₂O, technical grade (100% wt. |
| | actives) |
| CuCl2•2H2O | CuCl₂•2H₂O, technical grade (100% wt. |
| | actives) |
| Cu(OAcO)2•H2O | Cu(OAcO)₂•H₂O, technical grade (100% wt. |
| | actives) |
| Cu D-gluconate | Cu D-gluconate, technical grade (100% wt. |
| | actives) |
| Cu(I)Cl•H2O | Cu(I)Cl•H₂O, technical grade (100% wt. |
| | actives) |
| ethanol (100%) | ethanol, technical grade,(100% wt. active) |
| ethanol (95%) | ethanol, technical grade, (95% wt. active) |
| isopropanol | isopropanol, technical grade, (100% wt. |
| | active) |
| isobutanol | isobutanol, technical grade (100% wt. |
| | active) |
| n-propanol | n-propanol, technical grade,100% wt. |
| | actives |
| propylene glycol n- | propylene glycol n-propyl ether (100% wt. |
| propyl ether | active) supplied as Dowanol PnP (ex. DOW |
| | Chem. Co.) |
| phenol propane glycol | phenol propane glycol ether (100% wt. |
| ether | active) supplied as Dowanol PPH (ex. DOW |
| | Chem. Co.) |
| BTC-65 (50%) | C₁₂-C₁₆ alkyl dimethyl benzyl ammonium |
| | chloride provided in an aqueous alcoholic |
| | carrier (50% wt. actives) (ex. Stepan) |
| Onyxide 3300 (33%) | quaternary ammonium complex with |
| | saccharinate counterion, (33% wt. actives, |
| | balance ethanol) (ex. Stepan Co.) |
| Neodol 91-6 | nonionic surfactant, C₉-C₁₁ linear primary |
| | alcohol ethoxylate, avg. 6 mols. |
| | ethoxylation, 100% wt. actives (ex. Shell |
| | Chemical) |
| Steol C5370 | anionic surfactant, sodium laureth sulfate, 3 |
| | mols EO (70% wt. actives) (ex. Stepan Co.) |
| Glucopon 425N | alkylpolyglycoside (50% wt. actives) (ex. |
| | Cognis) |
| Plurafac SL62 | nonionic surfactant, linear alcohol |
| | ethoxylate (100% wt.active) (ex. BASF) |
| Tween 20 | sorbitan based surfactant |
| Silwet Hydrostable 212 | silylated/organic surfactant blend (used as |
| | supplied) (ex. Momentive Performance |
| | Materials, Inc.). |
| triethanolamine | triethanolamine, technical grade (100% wt. |
| | active) (ex. Dow Chemical Company) |
| ethanolamine | ethanolamine, technical grade (100% wt. |
| | active) (ex. Huntsman) |
| Citrasol 502 (50%) | aqueous citric acid solution (50% wt. active) |
| | (ex. ADM) |
| Citrosol 502(50%)/6 | aqueous solution of citric acid (50% wt. |
| | active) (ex. ADM) further diluted 1:5 in |
| | deionized water |
| NH4(OH) (29.86%) | aqueous solution of ammonium hydroxide |
| | (29.86% wt. active) |
| NaOH (50%) | aqueous solution of sodium hydroxide, |
| | 50% wt. active |
| NaOH (10%) | aqueous solution of sodium hydroxide |
| | (10% wt. active) |
| citric acid (1.56%) | aqueous solution of anhydrous laboratory |
| | grade citric acid (1.56% wt. active) |
| ammonium acetate | anhydrous solid, reagent grade (ex. JT |
| | Baker) (100% wt. actives) |
| Monacor BE | borate ester blend, used as supplied |
| | comprising MEA-borate ester and MIPA- |
| | borate ester (ex. Croda, tradename |
| | Crodacor BE) |
| IL2976 | corrosion inhibitor comprising mixed acid |
| | esters, a proprietary composition believed |
| | to be borate esters, used ‘as supplied’ |
| Trilon BX | aqueous solution of tetrasodium ethylene |
| | diamine tetraacetic acid 38.9% wt. actives) |
| | (ex. BASF) |
| di H₂O | deionized water, (100% wt. active) |

Further, wherein a specific composition was evaluated for microbicidal efficacy against a challenge microorganism according to one or more of the test protocols identified above, the results of these tests are reported as well. Wherein multiple challenge microorganisms were evaluated in any one test, multiple results are reported.

In the following tables, the tested microorganisms and their identity as reported on the tables are as indentified on Table B:

TABLE B

| | Microorganisms |
|---|---|
| Identifier | Type/Challenge microorganism |
| “PV1” | Virus/Poliovirus type 1 (Sabin), ex. supplied by U.S. Centers |
| | for Disease Control and Prevention (CDC) |
| “HAV” | Virus/Hepatitis A virus, strain MH-175 ex. Dr. Mark Sobsey, |
| | University of North Carolina, Chapel Hill, NC |
| “HAdV” | Virus/Human adenovirus type 5, supplied as ATCC VR-5 |
| “FCV” | Virus/Feline calicivirus strain F-9, supplied as ATCC VR-782 |
| “IV-A” | Virus/Influenza A virus, A/California/04/2009 (H1N1), supplied |
| | as Biodefence and Emerging Infections Research Resources |
| | Repository (BEI Resource) NR-13658 |
| “HSV” | Virus/Herpes simplex type 1, supplied as ATCC VR-1493 |
| “HRV” | Virus/Human rhinovirus type 14 strain 1059, supplied as |
| | ATCC VR-284 |
| “Rota” | Virus/Rotavirus Wa strain, supplied as ATCC VR-2018 |
| “M. bovis” or “Mb” | Bacteria/Mycobacterium bovis-BCG, supplied by Organon |
| | Teknika, Durham, NC to ATS Labs (used as a surrogate for |
| | testing efficacy against Tuberculosis) |
| “S. aureus” or “Sa” | Bacteria/Staphylococcus aureus, supplied as ATCC 6538 |
| “E. coli” or “Ec” | Bacteria/Escherichia coli, supplied as ATCC 10536 |
| “P. aeruginosa” or “Pa” | Bacteria/Pseudomonas aeruginosa (“P. aeruginosa”) |
| | (supplied as ATCC 15442); |
| “E. hirae”, or “Eh” | Bacteria/Enterococcus hirae, supplied as ATCC 10541 |
| “T. ment” | Fungus/Trichophyton metagropytes, supplied as ATCC 9533 |

The use of feline calicivirus strain was for its well known function as a ‘surrogate virus’ in place of Norovirus.

In the following tables, Table C describes various “comparative” examples, (which may be identified by the prepended letter “C”) while subsequent Table 1 describes various examples of inventive compositions according to the invention, (which may also be identified by the prepended letter “E”) as well as the observed physical properties and the results of microbidical testing according to one or more of the following standardized test protocols:

A) ASTM E1052-96(2002) Standard Test Method for Efficacy of Antimicrobial Agents against Viruses in Suspension, (for a 5 minute contact time, unless specified otherwise) identified on Table 1 as “ASTM E 1052 (log10 reduction)”;

B) ASTM E 1053-11 Standard Test Method to Assess Virucidal Activity of Chemicals Intended for Disinfection of Inanimate, Nonporous Environmental Surfaces, (for a 10 minute contact time, unless specified otherwise) identified on Table 1 as “ASTM 1053 (log10 reduction)”,

C) European Standard Surface Test, EN13697, identified on Table 1 as “EN 13697 (log10 reduction)”,

D) AOAC Germicidal Spray Products as Disinfectant Test Method, AOAC Index, 17th Ed. (2000), identified on Table 1 as “AOAC Germicidal Spray”. In this test, a result of “0/60” or “1/60” is equivalent to a result of “pass” according to that test's protocols. Results of “2” excess thereof for “/60” tested plates/samples are considered as being equivalent to a “fail” according to that test's protocols.

E) The European Standard Surface Test, EN13697 protocol was used for testing antifungal efficacy against Trichophytort mentagrophytes (supplied as ATCC 9533) the results reported on Tables C and 1 are the log10 reduction of the fungus.

It is noted that each tested composition was not necessarily tested according to all of the foregoing protocols as, test results of microbicidal efficacy against Poliovirus type 1 Sabin supports the presumption of efficacy against easier to control or eradicate microorganisms.

In the following tables the amount of the copper ions present are also indicated in parts per million (ppm) and this number is based on the empirical calculation of the available metal ions present in the indicated composition and 100% disassociation of the copper ion from the copper ion source is presumed for this empirical calculation.

In the following tables the appearance of the test compositions both of Table I and C are indicated; all of the liquid compositions were transparent unless indicated otherwise, e.g. “slight haze” and many were bluish in tint of a lesser or greater substantivity.

All of the compositions of both Table 1 and C were liquids which were readily pourable and pumpable and had a “water-thin” viscosity.

TABLE C
(Comparative Examples0

| | C1 | C2 | C3 | C4 | C5 | C6 |
|---|---|---|---|---|---|---|
| CuSO₄•5H₂O | — | 0.10 | 0.10 | 0.1 | 0.1 | 0.1 |
| ethanol (100%) | 35 | — | 35 | — | — | — |
| ethanol (95%) | — | — | — | — | — | — |
| BTC-65 (50%) | — | — | — | — | — | — |
| Onyxide 3300 (33%) | — | — | — | — | — | — |
| Neodol 91-6 | 0.18 | — | — | — | — | — |
| Plurafac SL62 | — | — | — | — | — | — |
| triethanolamine | 0.02 | 0.30 | — | — | — | 0.31 |
| Citrasol 502 (50%) | — | — | 0.055 | 0.04 | — | — |
| NH4(OH) (29.86%) | — | — | — | — | — | 0.12 |
| di H₂O | 64.88 | 99.56 | 64.80 | 99.86 | 99.90 | 99.49 |
| pH | 9.08 | 8.08 | 3.08 | 3.08 | 5.08 | 9.22 |
| copper ion content (ppm) | — | 254 | 254 | 254 | 254 | 254 |
| appearance | colorless | medium blue | colorless | colorless | colorless | colorless |
| ASTM E 1052 (log10 reduction) | PV1 ≦ 1.17 | PV1 ≦ 1.50 | PV1 ≦ 2.44 | PV1 < 1.83 | PV1 < 1.67 | PV1 < 1.67 |
| | | IV-A< 1.33 | | | FCV < 1.83 | |
| | | IV-A < 0.33 | | | | |
| | | HSV < 2.17 | | | | |
| | | HAdV < 1.73 | | | | |
| ASTM 1053 (log10 reduction) | — | — | — | — | — | — |
| AOAC Germicidal Spray | — | Sa = 60/60 | — | — | — | — |
| | | (fail) | | | | |
| | | Pa = 23/60 | | | | |
| | | (fail) | | | | |
| EN 13697 (log10 reduction) | Sa > 6.55 | Sa < 1.97 | — | — | — | — |
| | Ec > 5.50 | Ec = 0.44 | | | | |
| | Pa > 4.52 | Pa = 0.78 | | | | |
| | Eh > 6.68 | | | | | |
| T.ment (log10 reduction) | — | 0.06 | — | — | — | — |

| | C7 | C8 | C9 | C10 | C11 | C12 | C13 |
|---|---|---|---|---|---|---|---|
| CuSO₄•5H₂O | 0.1 | — | — | — | — | — | — |
| ethanol (100%) | — | 35 | 35 | 35 | 35 | 50 | 50 |
| ethanol (95%) | — | — | — | — | — | — | — |
| BTC-65 (50%) | — | — | — | — | — | — | — |
| Onyxide 3300 (33%) | — | — | — | — | — | — | — |
| Neodol 91-6 | — | — | — | — | — | 0.1 | — |
| Plurafac SL62 | — | — | — | — | — | — | — |
| triethanolamine | 0.31 | — | 0.002 | 0.08 | — | 0.014 | 0.015 |
| Citrasol 502 (50%) | — | 0.30 | 0.02 | 0.016 | 0.016 | — | — |
| NH4(OH) (29.86%) | 0.08 | — | — | — | — | — | — |
| NaOH (10%) | 0.39 | — | — | — | 0.01 | — | — |
| di H₂O | 99.1 | 64.7 | 64.98 | 64.9 | 64.8 | 49.88 | 49.98 |
| pH | 10.95 | 3.07 | 5.01 | 9.01 | 11.15 | 8.87 | 8.98 |
| copper ion content (ppm) | 254 | — | — | — | — | — | — |
| appearance | colorless | colorless | colorless | colorless | colorless | colorless | colorless |
| ASTM E 1052 (log10 reduction) | PV1 < 1.42 | PV1 ≦ 1.77 | PV1 < 1.67 | PV1 ≦ 2.10 | PV < 1.67 | PV1 ≦ 2.00 | PV1 ≦ 1.5 |
| | FCV > 3.33 | | FCV < 2.33 | | FCV > 3.83 | | |
| ASTM 1053 (log10 reduction) | — | — | — | — | — | — | — |
| AOAC Germicidal Spray | — | — | — | — | — | — | — |
| EN 13697 (log10 reduction) | — | — | — | — | — | — | — |
| T.ment (log10 reduction) | — | — | — | — | — | — | — |
| | C14 | C15 | C16 | C17 | C18 | C19 | C20 |
| CuSO₄•5H₂O | — | — | — | — | 0.1 | — | — |
| ethanol (100%) | 20.0 | — | — | 10 | 10 | 10 | 10 |
| ethanol (95%) | — | — | — | — | — | — | — |
| isobutanol | — | 35.0 | — | — | — | — | — |
| isopropanol | — | — | 35 | — | — | — | — |
| n-propanol | — | — | — | — | — | — | — |
| BTC-65 (50%) | — | — | — | — | — | — | — |
| Onyxide 3300 (33%) | — | — | — | — | — | — | — |
| Neodol 91-6 | — | — | — | — | — | — | — |
| Plurafac SL62 | — | — | — | — | — | — | — |
| triethanolamine | 0.003 | 0.003 | 0.003 | — | — | — | — |
| Citrasol 502 (50%) | — | — | — | 0.085 | 0.04 | 0.0025 | 0.02 |
| NH4(OH) (29.86%) | — | — | — | — | — | 0.0035 | — |
| NaOH (10%) | — | — | — | — | — | — | — |
| di H₂O | 80.0 | 65.0 | 65.0 | 89.9 | 89.9 | 89.99 | 89.98 |
| pH | 8.81 | 8.93 | 8.47 | 3.07 | 3.03 | 5.07 | 9.22 |
| copper ion content (ppm) | — | — | — | — | 256 | — | — |
| appearance | colorless | light blue, | colorless | colorless | colorless | colorless | colorless |
| | | two phases | | | | | |
| ASTM E 1052 (log10 reduction) | PV1 < 0.73 | PV1 < 0.83 | PV1 < 0.56 | PV1 < 0.73 | PV1 < 0.46 | PV1 < 0.73 | PV1 < 0.90 |
| ASTM 1053 (log10 reduction) | — | — | — | — | — | — | |
| AOAC Germicidal Spray | — | — | — | — | — | — | — |
| EN 13697 (log10 reduction) | Sa < 1.02 | — | — | — | — | — | — |
| | EC < 1.20 | | | | | | |
| | Pa = 0.00 | | | | | | |
| | Eh < 1.36 | | | | | | |
| T.ment (log10 reduction) | <0.22 | — | — | — | — | — | — |
| | C21 | C22 | C23 | C24 | C25 | C26 | C27 |
| CuSO₄•5H₂O | — | — | — | — | 0.099 | — | 0.099 |
| ethanol (100%) | 10 | 12 | — | 17.5 | — | — | — |
| ethanol (95%) | — | — | — | — | — | — | — |
| isobutanol | — | 12 | — | 17.5 | — | — | — |
| isopropanol | — | — | — | — | — | — | — |
| n-propanol | — | 12 | 35 | — | — | — | — |
| propylene glycol n-propyl | — | — | — | — | 50.0 | 50 | — |
| ether | | | | | | | |
| phenol propane glycol ether | — | — | — | — | — | — | 50 |
| BTC-65 (50%) | — | — | — | — | — | — | — |
| Onyxide 3300 (33%) | — | — | — | — | — | — | — |
| Neodol 91-6 | — | — | — | — | — | — | — |
| Plurafac SL62 | — | — | — | — | — | — | — |
| triethanolamine | 0.15 | 0.003 | 0.003 | — | 0.40 | 0.003 | 0.40 |
| Citrasol 502 (50%) | — | — | — | — | — | — | — |
| NH4(OH) (29.86%) | — | — | — | — | — | — | — |
| NaOH (10%) | 0.035 | — | — | — | — | — | — |
| di H₂O | 89.98 | 64.0 | 65.0 | 65.0 | 49.5 | 50 | 49.5 |
| pH | 11.04 | 8.21 | 8.63 | 8.36 | 7.95 | 8.26 | 8.02 |
| copper ion content (ppm) | — | — | — | — | 254 | — | 254 |
| appearance | colorless | colorless | colorless | colorless | light blue | colorless | light blue, |
| | | | | | | | two phase |
| ASTM E 1052 (log10 reduction) | PV1 < 0.56 | PV1 < 0.50 | PV1 < 0.50 | PV1 = 1.17 | PV1 < 0.17 | PV1 < 0.17 | PV1 < 0.17 |
| ASTM 1053 (log10 reduction) | — | — | — | — | — | — | — |
| AOAC Germicidal Spray | — | — | — | — | — | — | — |
| EN 13697 (log10 reduction) | — | — | — | — | — | — | — |
| T.ment (log10 reduction) | — | — | — | — | — | — | — |

| | C28 | C29 | C30 |
|---|---|---|---|
| CuSO₄•5H₂O | — | — | 0.1 |
| ethanol (100%) | — | 45 | — |
| ethanol (95%) | — | — | — |
| BTC-65 (50%) | 0.2 | 0.2 | — |
| Onyxide 3300 (33%) | — | — | — |
| Neodol 91-6 | 0.1 | 0.1 | 0.1 |
| triethanolamine | 0.022 | 0.04 | 0.39 |
| di H₂O | 99.67 | 54.66 | 99.4 |
| pH | 9 | 9.09 | 8.08 |
| copper ion content (ppm) | 0 | 0 | 254 |
| appearance | colorless | colorless | light blue |
| ASTM E 1052 (log10 reduction) | PV1 =< 3.00 | PV1 < 2.67 | PV1 =< 2.5 |
| | IV-A > 5.00 | PV1 < 217 | IV-A > 6.00 |
| | HSV > 6.00 | PV1 < 1.53 | IV-A > 5.17 |
| | HAdV < 2.0 | HSV > 6.00 | HSV > 7.5 |
| | | HAdV > 4.5 | HSV > 7.0 |
| | | HRV =< 3.83 | HAdV < 1.0 |
| ASTM 1053 (log10 reduction) | — | — | — |
| AOAC Germicidal Spray | — | — | Sa = 57/60 |
| | | | Pa = 27/60 |
| EN 13697 (log10 reduction) | Sa = 4.31 | — | Sa < 1.17 |
| | EC > 5.50 | | Ec = 2.12 |
| | Pa > 4.52 | | Pa = 2.84 |
| | Eh = 5.48 | | Eh < 1.3 |
| T.ment (log10 reduction) | — | — | — |

| | C31 | C32 | C33 | C34 | C35 |
|---|---|---|---|---|---|
| CuSO₄•5H₂O | 0.1 | — | 0.1 | — | 0.1 |
| ethanol (100%) | 5 | 5 | 10 | 10 | 50 |
| ethanol (95%) | — | — | — | — | — |
| BTC-65 (50%) | 0.2 | 0.2 | 0.2 | 0.2 | 0.2 |
| Onyxide 3300 (33%) | — | — | — | — | — |
| Neodol 91-6 | 0.1 | 0.1 | 0.1 | 0.1 | 0.1 |
| Plurafac SL62 | — | — | — | — | — |
| triethanolamine | 0.31 | — | 0.31 | — | — |
| citric acid (1.56%) | — | — | — | — | — |
| di H₂O | 94.3 | 94.7 | 89.3 | 89.70 | 49.60 |
| pH | 8.04 | 8.44 | 7.97 | 8.35 | 4.74 |
| copper ion content (ppm) | 254 | 0 | 254 | 0 | — |
| appearance | medium blue | colorless | light blue | colorless | — |
| ASTM E 1052 (log10 reduction) | PV1 =< 0.23 | PV1 < 0.23 | PV1 < 0.9 | PV1 < 0.23 | — |
| | IV-A > 5.00 | | | | |
| | HSV > 6.00 | | | | |
| ASTM 1053 (log10 reduction) | — | — | — | — | HRV = 1.50 |
| AOAC Germicidal Spray | Sa = 0/30 | — | — | — | — |
| | Pa = 0/30 | | | | |
| EN 13697 (log10 reduction) | Sa = 3.48 | Sa > 6.40 | — | — | — |
| | Ec > 6.58 | Ec > 6.58 | | | |
| | Pa > 5.16 | Pa > 5.16 | | | |
| | Eh = 3.43 | Eh > 6.74 | | | |
| T.ment (log10 reduction) | — | — | — | — | — |

| | C36 | C37 | C38 | C39 |
|---|---|---|---|---|
| CuSO₄•5H₂O | — | — | — | — |
| ethanol (100%) | 50 | 35 | 35 | — |
| ethanol (95%) | — | — | — | — |
| BTC-65 (50%) | 0.2 | 0.2 | — | 0.2 |
| Onyxide 3300 (33%) | — | — | — | — |
| Neodol 91-6 | 0.1 | 0.1 | — | — |
| triethanolamine | — | 0.016 | 0.025 | — |
| di H₂O | 0.012 | 64.68 | 64.97 | 99.8 |
| pH | 49.68 | 8.89 | 9.23 | 8.58 |
| copper ion content (ppm) | 8.64 | 0 | 0 | 0 |
| appearance | 0 | clear | clear | clear |
| ASTM E 1052 (log10 reduction) | colorless | PV1 =< 2.17 | PV1 =< 1.83 | PV1 =< 1.83 |
| | | | FCV > 6.00 | IV-A > 5.00 |
| | | | | HSV > 6.00 |
| | | | | HAdv < 1.5 |
| ASTM 1053 (log10 reduction) | PV1 < 1.25 | — | — | — |
| | HAdv > 4.50 | | | |
| | HRV > 6.00 | | | |
| | FCV > 4.33 | | | |
| | FCV > 6.00 | | | |
| AOAC Germicidal Spray | — | — | — | Sa = 1/60 |
| | | | | PA = 1/60 |
| EN 13697 (log10 reduction) | — | Sa > 6.35, | Sa = 4.60, | Sa = 4.47, |
| | | Ec > 5.35, | Ec = 4.19, | Ec = 4.28, |
| | | Pa > 3.85 | Pa > 4.52 | Pa => 4.52 |
| | | | Eh > 6.68 | Eh = 3.83 |
| T.ment (log10 reduction) | Sa > 6.12 | — | 4.84 | 2.73 |
| | Ec > 5.66 | | | |
| | Pa > 3.80 | | | |
| | Eh > 6.50 | | | |
| — | >5.74 | — | — | — |

TABLE 1
(Examples)

| | E1 | E2 | E3 | E4 | E5 | E6 | E7 |
|---|---|---|---|---|---|---|---|
| CuSO₄•5H₂O | 0.1 | 0.1 | 0.1 | 0.1 | 0.1 | 0.1 | 0.1 |
| ethanol (100%) | 35 | 35 | 50 | 50 | 35 | 35 | 35 |
| ethanol (95%) | — | — | — | — | — | — | — |
| BTC-65 (50%) | — | — | — | — | — | — | — |
| Onyxide 3300 | — | — | — | — | — | — | — |
| (33%) | | | | | | | |
| Neodol 91-6 | 0.10 | — | 0.10 | — | — | — | — |
| triethanolamine | 0.41 | 0.41 | 0.34 | 0.45 | 0.23 | 0.97 | 0.20 |
| Citrasol 502 | — | — | — | — | 0.11 | — | 0.02 |
| (50%) | | | | | | | |
| NH4(OH) | — | — | — | — | — | 0.10 | 0.10 |
| (29.86%) | | | | | | | |
| NaOH (10%) | — | — | — | — | — | — | 0.37 |
| di H₂O | 64.39 | 64.49 | 49.46 | 49.45 | 64.6 | 63.8 | 64.2 |
| pH | 8.09 | 8.10 | 8.01 | 8.11 | 5.10 | 8.95 | 10.98 |
| copper ion | 254 | 254 | 254 | 254 | 254 | 254 | 254 |
| content (ppm) | | | | | | | |
| appearance | light blue | light blue | light blue | light blue | colorless | colorless | colorless |
| ASTM E 1052 | PV1 < 2.23 | PV1 + 2.50 | PV1 < 3.48 | PV1 ≦ 3.00 | PV1 < 1.5 | PV1 < 2.27 | PV1 < 3.67 |
| (log10 | | | | PV1 ≦ 4.09 | | | |
| reduction) | | | | | | | |
| ASTM 1053 | — | — | — | — | — | — | — |
| (log10 | | | | | | | |
| reduction) | | | | | | | |
| AOAC | Sa = 0/60 | — | Sa = 0/60 | — | — | — | — |
| Germicidal | Pa = 0/60 | | Pa = 0/60 | | | | |
| Spray | | | | | | | |
| EN 13697 | Sa > 6.23 | — | Sa > 6.23 | — | — | — | — |
| (log10 | Ec > 5.50 | | Ec > 5.50 | | | | |
| reduction) | Pa > 3.38 | | Pa > 3.38 | | | | |
| | Eh > 6.36 | | Eh > 6.36 | | | | |
| T.ment (log10 | — | — | >5.74 | — | — | — | — |
| reduction) | | | | | | | |

| | E8 | E9 | E10 | E11 | E12 | E13 |
|---|---|---|---|---|---|---|
| CuSO₄•5H₂O | 0.1 | 0.1 | 0.1 | 0.1 | 0.1 | 0.1 |
| ethanol (100%) | 50 | 50 | 20 | — | — | 10 |
| ethanol (95%) | — | — | — | — | — | — |
| isopropanol | — | — | — | 35 | — | — |
| isobutanol | — | — | — | — | 35 | — |
| BTC-65 (50%) | — | — | — | — | — | — |
| Onyxide 3300 | — | — | — | — | — | — |
| (33%) | | | | | | |
| Neodol 91-6 | 0.1 | — | — | — | — | — |
| triethanolamine | 0.34 | 0.45 | 0.45 | 0.45 | 0.40 | — |
| Citrasol 502 | — | — | — | — | — | — |
| (50%) | | | | | | |
| NH4(OH) | 0.1 | 0.1 | — | — | — | — |
| (29.86%) | | | | | | |
| NaOH (10%) | — | — | — | — | — | — |
| di H₂O | 49.4 | 49.3 | 79.4 | 64.4 | 64.5 | 89.9 |
| pH | 10 | 10 | 8.12 | 8.0 | 8.08 | 5.01 |
| copper ion | 254 | 254 | 254 | 254 | 254 | 254 |
| content (ppm) | | | | | | |
| appearance | light blue | light blue | colorless | colorless | light blue, two | colorless |
| | | | | | phases | |
| ASTM E 1052 | PV1 > 3.73 | PV1 > 3.73 | PV < 2.10 | PV < 0.23 | PV1 < 1.33 | PV1 < 0.73 |
| (log10 | | | | | | |
| reduction) | | | | | | |
| ASTM 1053 | PV1 > 4.50 | — | — | — | — | — |
| (log10 | | | | | | |
| reduction) | | | | | | |
| AOAC | Sa = 0/60 | — | — | — | — | — |
| Germicidal | (pass) | | | | | |
| Spray | Pa = 0/60 | | | | | |
| | (pass) | | | | | |
| EN 13697 | — | — | — | — | — | — |
| (log10 | | | | | | |
| reduction) | | | | | | |
| T.ment (log10 | — | — | — | — | — | — |
| reduction) | | | | | | |
| | E14 | E15 | E16 | E17 | E18 | E19 |
| CuSO₄•5H₂O | 0.1 | 0.1 | 0.099 | 0.1 | 0.099 | 0.099 |
| ethanol (100%) | 10 | 10 | 12.0 | — | 17.5 | 35.0 |
| ethanol (95%) | — | — | — | — | — | — |
| isopropanol | — | — | 12.0 | — | — | — |
| n-propanol | — | — | 12.0 | 35.0 | — | — |
| isobutanol | — | — | — | — | 17.5 | — |
| propylene | — | — | — | — | — | 25.0 |
| glycol | | | | | | |
| n-propyl ether | | | | | | |
| BTC-65 (50%) | — | — | — | — | — | — |
| Onyxide 3300 | — | — | — | — | — | — |
| (33%) | | | | | | |
| Neodol 91-6 | — | — | — | — | — | — |
| triethanolamine | 0.30 | 0.30 | 0.40 | 0.50 | 0.40 | 0.40 |
| Citrasol 502 | — | — | — | — | — | — |
| (50%) | | | | | | |
| NH4(OH) | 0.14 | 0.12 | — | — | — | — |
| (29.86%) | | | | | | |
| NaOH (10%) | — | 0.35 | — | — | — | — |
| di H₂O | 89.5 | 89.1 | 63.5 | 64.4 | 64.5 | 39.5 |
| pH | 9.07 | 10.97 | 7.89 | 8.03 | 7.90 | 7.89 |
| copper ion | 254 | 254 | 254 | 254 | 254 | 254 |
| content (ppm) | | | | | | |
| appearance | colorless | colorless | light blue | colorless | light blue | light blue |
| ASTM E 1052 | PV1 ≦ 2.10 | PV1 < 0.73 | PV1 < 0.67 | PV1 < 1.33 | PV1 < 1.33 | PV1 < 0.50 |
| (log10 | FCV < 1.00 | | | | | |
| reduction) | | | | | | |
| ASTM 1053 | — | — | — | — | — | — |
| (log10 | | | | | | |
| reduction) | | | | | | |
| AOAC | — | — | — | — | — | — |
| Germicidal | | | | | | |
| Spray | | | | | | |
| EN 13697 | — | — | — | — | — | — |
| (log10 | | | | | | |
| reduction) | | | | | | |
| T.ment (log10 | — | — | — | — | — | — |
| reduction) | | | | | | |

| | E20 | E21 | E22 | E23 | E24 | E25 | E26 | E27 |
|---|---|---|---|---|---|---|---|---|
| CuSO₄•5H₂O | 0.1 | 0.1 | 0.199 | 0.1 | 0.1 | 0.1 | 0.1 | 0.1 |
| ethanol (100%) | 25 | 45 | 45 | 75 | 35 | 35 | 50 | 50 |
| ethanol (95%) | — | — | — | — | — | — | — | — |
| BTC-65 (50%) | 0.2 | 0.2 | — | 0.2 | 0.2 | 0.2 | 0.2 | 0.2 |
| Onyxide 3300 | — | — | 0.30 | — | — | — | — | — |
| (33%) | | | | | | | | |
| Neodol 91-6 | 0.1 | 0.1 | 0.1 | 0.1 | 0.1 | 0.1 | 0.1 | 0.1 |
| triethanolamine | 0.42 | 0.19 | 0.3 | 0.302 | 0.443 | — | — | 0.37 |
| ethanolamine | — | — | — | — | — | — | — | — |
| NH4OH | — | 0.1 | 0.09 | — | — | — | — | — |
| (29.8%) | | | | | | | | |
| NaOH (10%) | — | — | — | — | — | — | — | — |
| Citrosol | — | — | — | — | — | — | — | — |
| 502(50%) | | | | | | | | |
| ammonium | — | — | — | — | — | 2.0 | 2.0 | — |
| acetate | | | | | | | | |
| Monacor BE | — | 0.1 | 0.1 | — | — | — | — | — |
| Trilon BX | — | — | — | — | — | — | — | — |
| di H₂O | 74.18 | 54.21 | 53.907 | 24.298 | 64.157 | 62.6 | 47.6 | 49.22 |
| pH | 8.09 | 9.5 | 8.85 | 8.23 | 8.06 | 6.82 | 7.08 | 8.06 |
| copper ion | 254 | 254 | 506 | 254 | 254 | 254 | 254 | 254 |
| content (ppm) | | | | | | | | |
| appearance | colorless | light blue | light blue | light blue | light blue | light blue | light blue | light blue |
| ASTM E 1052 | PV1 > 3.67 | PV1 > 5.27 | PV1 > | PV1 > 5.67 | PV1 > 4.87 | — | — | PV1 > 5.89 |
| (log10 | | PV1 > 5.88 | 5.67 | | FCV > 6.00 | | | HAdV > 5.00 |
| reduction) | | HAV > 6.00 | | | | | | HRV > 6.00 |
| | | FCV > 6.00 | | | | | | HAV > 6.00 |
| | | | | | | | | FCV > 6.00 |
| ASTM 1053 | — | 1 minute: | — | — | — | PV1 > 4.38 | PV1 > | PV1 > 3.50 |
| (log10 | | PV1 > 4.50 | | | | HRV > 2.75 | 3.50 | PV1 > 4.50 |
| reduction) | | 5 minutes: | | | | Rota > 2.5 | | |
| | | PV1 > 4.50 | | | | | | |
| AOAC | — | Sa = 0/60 | — | — | — | — | — | Sa = 0/30 |
| Germicidal | | Pa = 0/60 | | | | | | Pa = 0/30 |
| Spray Test | | Mb = 0/30 | | | | | | |
| | | (hard surface | | | | | | |
| | | test) and | | | | | | |
| | | ≦5.32 | | | | | | |
| | | (suspension | | | | | | |
| | | test) | | | | | | |
| EN 13697 | — | — | — | — | — | — | — | Sa > 6.29, |
| (log10 | | | | | | | | Ec > 5.90, |
| reduction) | | | | | | | | Pa > 4.23, |
| | | | | | | | | Eh > 5.67 |
| T.ment (log10 | — | — | — | — | — | — | — | >5.74 |
| reduction) | | | | | | | | |

| | E28 | E29 | E30 | E31 | E32 |
|---|---|---|---|---|---|
| CuSO₄•5H₂O | 0.1 | 0.1 | 0.1 | 0.1 | 0.1 |
| ethanol (100%) | 40 | 40 | 40 | 40 | 40 |
| ethanol (95%) | — | — | — | — | — |
| BTC-65 (50%) | — | — | — | — | — |
| Onyxide 3300 | 0.303 | 0.303 | 0.303 | 0.303 | 0.303 |
| (33%) | | | | | |
| Neodol 91-6 | 0.1 | 0.1 | 0.1 | 0.1 | 0.1 |
| triethanolamine | 0.13 | 0.13 | 0.13 | 0.13 | 0.13 |
| ethanolamine | 0.05 | 0.05 | 0.05 | 0.05 | 0.05 |
| NH4OH | 0.12 | 0.12 | 0.12 | 0.12 | 0.12 |
| (29.8%) | | | | | |
| NaOH (10%) | — | — | — | — | — |
| Citrosol | — | — | — | — | — |
| 502(50%) | | | | | |
| ammonium | — | — | — | — | — |
| acetate | | | | | |
| Monacor BE | — | — | 0.20 | — | — |
| Trilon BX | — | — | — | — | — |
| IL2976 | 0.060 | 0.20 | — | 1.22 | 0.11 |
| di H₂O | 58.6 | 59.0 | 59.0 | 58.0 | 59.1 |
| pH | 9.10 | 9.10 | 9.59 | 9.50 | 9.10 |
| copper ion | 254 | 254 | 254 | 254 | 254 |
| content (ppm) | | | | | |
| appearance | light blue | light blue | light blue | light blue | light blue |
| ASTM E 1052 | PV1 =< 3.44 | PV1 =< 3.54 | PV1 =< 5.10 | PV1 =< 3.10 | PV1 =< 2.44 |
| (log10 | | | | | |
| reduction) | | | | | |
| ASTM 1053 | — | — | — | — | — |
| (log10 | | | | | |
| reduction) | | | | | |
| AOAC | — | — | — | — | — |
| Germicidal | | | | | |
| Spray Test | | | | | |
| EN 13697 | — | — | — | — | — |
| (log10 | | | | | |
| reduction) | | | | | |
| T.ment (log10 | — | — | — | — | — |
| reduction) | | | | | |
| | E33 | E34 | E35 | E36 | E37 |
| CuSO₄•5H₂O | 0.1 | 0.1 | 0.1 | 0.1 | 0.1 |
| ethanol (100%) | 50 | 50 | 50 | 50 | 50 |
| ethanol (95%) | — | — | — | — | — |
| BTC-65 (50%) | 0.2 | 0.2 | 0.2 | 0.2 | 0.2 |
| Onyxide 3300 | — | — | — | — | — |
| (33%) | | | | | |
| Neodol 91-6 | 0.1 | — | 0.1 | 0.1 | 0.1 |
| triethanolamine | 0.48 | 0.37 | 0.41 | — | 0.48 |
| ethanolamine | — | — | — | — | — |
| NH4OH | — | — | — | — | — |
| (29.8%) | | | | | |
| NaOH (50%) | — | — | 0.054 | 0.03 | — |
| Citrosol | — | — | q.s. | — | — |
| 502(50%) | | | | | |
| sodium citrate | — | — | — | 0.1 | — |
| ammonium | — | — | — | — | — |
| acetate | | | | | |
| Monacor BE | — | — | — | — | 0.06 |
| di H₂O | 49.12 | 49.32 | 49.45 | 49.57 | 49.06 |
| pH | 8.06 | 8.10 | 8.24 | 8.45 | 8.52 |
| copper ion | 254 | 254 | 254 | 254 | 254 |
| content (ppm) | | | | | |
| appearance | light blue | light blue | cloudy | colorless | — |
| ASTM E 1052 | PV1 > 5.00 | PV1 = 0.73 | PV1 =< 5.00 | PV1 > 5.67 | — |
| (log10 | | | | | |
| reduction) | | | | | |
| ASTM 1053 | PV1 > 3.50 | — | — | — | — |
| (log10 | PV1 > 4.50 | | | | |
| reduction) | | | | | |
| AOAC | — | — | — | — | — |
| Germicidal | | | | | |
| Spray Test | | | | | |
| EN 13697 | — | — | — | — | — |
| (log10 | | | | | |
| reduction) | | | | | |
| | E38 | E39 | E40 | E41 | E42 |
| CuSO₄•5H₂O | 0.1 | 0.1 | 0.1 | 0.1 | 0.1 |
| ethanol (100%) | — | 45 | 45 | 25 | 45 |
| ethanol (95%) | 50 | — | — | — | — |
| isopropanol | — | — | — | 20 | — |
| BTC-65 (50%) | 0.1 | — | — | 0.2 | 0.2 |
| Onyxide 3300 | — | 0.3 | 0.3 | — | — |
| (33%) | | | | | |
| Neodol 91-6 | 0.1 | 0.1 | 0.1 | 0.1 | 0.1 |
| Steol CS370 | — | — | — | — | 0.143 |
| triethanolamine | 0.31 | 0.48 | 0.18 | 0.4 | 0.43 |
| ethanolamine | — | — | — | — | — |
| NH4OH | — | — | 0.11 | — | — |
| (29.8%) | | | | | |
| NaOH (10%) | — | — | — | — | — |
| Citrosol | — | — | — | — | — |
| 502(50%) | | | | | |
| Citrosol | — | — | — | — | — |
| 502(50%)/6 | | | | | |
| ammonium | — | — | — | — | — |
| acetate | | | | | |
| Monacor BE | — | — | — | — | — |
| Trilon BX | — | — | — | — | — |
| di H₂O | 49.39 | 54.02 | 54.21 | 54.2 | 54.027 |
| pH | 8.1 | 8.2 | 9.17 | 8.03 | 8.08 |
| copper ion | 254 | 254 | 254 | 254 | 254 |
| content (ppm) | | | | | |
| appearance | light blue | light blue | light blue | light blue | dark blue, |
| | | | | | slight haze |
| ASTM E 1052 | — | PV1 > 5.27 | PV1 > 4.50 | PV1 = 4.83 | PV1 > 6.00 |
| (log10 | | HRV > 4.83 | | | |
| reduction) | | HSV > 6.00 | | | |
| ASTM 1053 | — | — | PV1 > 4.50 (at | — | — |
| (log10 | | | 1 min), | | |
| reduction) | | | PV1 > 4.50 (at | | |
| | | | 5 min) | | |
| AOAC | — | — | Sa = 0/60 | — | — |
| Germicidal | | | Pa = 0/60 | | |
| Spray Test | | | | | |
| EN 13697 | — | — | — | — | — |
| (log10 | | | | | |
| reduction) | | | | | |

| | E43 | E44 | E45 | E46 | E47 | E48 |
|---|---|---|---|---|---|---|
| CuSO₄•5H₂O | 0.1 | 0.1 | 0.1 | 0.1 | 0.1 | 0.1 |
| ethanol (100%) | 45 | — | 45 | 45 | 45 | 45 |
| ethanol (95%) | — | — | — | — | — | — |
| isopropanol | — | — | — | — | — | — |
| n-propanol | — | 45 | — | — | — | — |
| BTC-65 (50%) | 0.2 | 0.2 | 0.2 | — | — | 0.2 |
| Onyxide 3300 | — | — | — | — | — | — |
| (33%) | | | | | | |
| BTC-2125M | — | — | — | 0.2 | — | — |
| BTC-1010 | — | — | — | — | 0.2 | — |
| Neodol 91-6 | 0.1 | 0.1 | — | 0.1 | 0.1 | — |
| Steol CS370 | — | — | 0.143 | — | — | — |
| Glucopon 425N | — | — | — | — | — | 0.2 |
| triethanolamine | 0.41 | 0.4 | 0.41 | 0.42 | 0.46 | 0.42 |
| ethanolamine | — | — | — | — | — | — |
| NH4OH | — | — | — | — | — | — |
| (29.8%) | | | | | | |
| NaOH (10%) | — | — | — | — | — | — |
| Citrosol | — | — | — | — | — | — |
| 502(50%) | | | | | | |
| ammonium | — | — | — | — | — | — |
| acetate | | | | | | |
| Monacor BE | — | — | — | — | — | — |
| Trilon BX | — | — | — | — | — | — |
| di H₂O | 54.19 | 54.2 | 54.14 | 54.18 | 54.14 | 54.08 |
| pH | 8.09 | 8.09 | 8.11 | 8.12 | 8.12 | 8.13 |
| copper ion | 254 | 254 | 254 | 254 | 254 | 254 |
| content (ppm) | | | | | | |
| appearance | light blue | — | dark blue, | light blue | light blue | dark blue |
| | | | slight | | | |
| | | | haze | | | |
| ASTM E 1052 | PV1 > 6.33 | PV1 < 3.00 | PV1 > 6.00 | PV1 > 6.00 | PV1 > 6.00 | PV1 > 6.00 |
| (log10 | HRV > 6.00 | | | | | |
| reduction) | | | | | | |
| ASTM 1053 | — | — | — | — | — | — |
| (log10 | | | | | | |
| reduction) | | | | | | |
| AOAC | Sa = 0/30 | — | — | — | — | — |
| Germicidal | Pa = 0/30 | | | | | |
| Spray Test | | | | | | |
| EN 13697 | — | — | — | — | — | — |
| (log10 | | | | | | |
| reduction) | | | | | | |

| | E49 | E50 | E51 | E52 | E53 | E54 | E55 | E56 |
|---|---|---|---|---|---|---|---|---|
| CuSO₄•5H₂O | 0.1 | 0.1 | 0.1 | 0.1 | 0.1 | 0.1 | 0.1 | 0.1 |
| ethanol (100%) | 45 | 45 | 40 | 38 | 45 | 38 | 38 | 38 |
| ethanol (95%) | — | — | — | — | — | — | — | — |
| isopropanol | — | — | — | — | — | — | — | — |
| n-propanol | — | — | — | — | — | — | — | — |
| BTC-65 (50%) | 0.2 | 0.2 | 0.2 | 0.2 | 0.2 | 0.2 | 0.2 | 0.2 |
| Onyxide 3300 | — | — | — | — | — | — | — | — |
| (33%) | | | | | | | | |
| BTC-2125M | — | — | — | — | — | — | — | — |
| Neodol 91-6 | — | — | 0.1 | 0.1 | 0.1 | 0.1 | 0.1 | 0.1 |
| Steol CS370 | — | — | — | — | — | — | — | — |
| Ammonyx LO | 0.33 | — | — | — | — | — | — | — |
| Plurafac SL62 | — | 0.1 | — | — | — | — | — | — |
| triethanolamine | 0.48 | 0.43 | 0.42 | 0.015 | 0.0102 | 0.42 | 0.08 | — |
| ethanolamine | — | — | — | — | — | — | — | — |
| NH4OH | — | — | — | — | — | — | 0.11 | 0.11 |
| (29.8%) | | | | | | | | |
| NaOH (10%) | — | — | — | — | — | — | — | — |
| Citrosol | — | — | — | 0.08 | 0.066 | — | — | — |
| 502(50%) | | | | | | | | |
| ammonium | — | — | — | — | — | — | — | — |
| acetate | | | | | | | | |
| Monacor BE | — | — | — | — | — | — | — | — |
| Trilon BX | — | — | — | — | — | — | — | — |
| di H₂O | 53.88 | 54.17 | 59.18 | 61.58 | 54.4 | 61.18 | 61.41 | 61.49 |
| pH | 8.18 | 8.14 | 8.10 | 5.0 | 5.04 | 8.08 | 9.20 | 9.38 |
| copper ion | 254 | 254 | 254 | 254 | 254 | 254 | 254 | 254 |
| content (ppm) | | | | | | | | |
| appearance | dark blue | light blue | light blue | light blue | light blue | light blue | light blue | — |
| ASTM E 1052 | PV1 > 6.00 | PV1 > 6.00 | PV1 > | PV1 > 5.62 | PV1 => 4.73 | PV1 = 4.54 | PV1 > | — |
| (log10 | | | 5.69 | PV1 > 4.73 | | HRV > 6.00 | 5.65 | |
| reduction) | | | HRV > | HAV => 6.00 | | | HAV > | |
| | | | 6.00 | HAdV > 4.50 | | | 6.00 | |
| | | | | FCV => 3.33 | | | FCV > | |
| | | | | | | | 6.00 | |
| ASTM 1053 | — | — | — | — | PV1 => 3 | — | — | — |
| (log10 | | | | | (5 min, | | | |
| reduction) | | | | | aluminum | | | |
| | | | | | Can, | | | |
| | | | | | complete | | | |
| | | | | | inactivation) | | | |
| | | | | | PV1 = 3.5 | | | |
| | | | | | (5 min, glass | | | |
| | | | | | jar | | | |
| | | | | | pressured, | | | |
| | | | | | completed | | | |
| | | | | | inactivation) | | | |
| AOAC | — | — | — | — | — | — | Sa = 0/30 | — |
| Germicidal | | | | | | | Pa = 0/30 | |
| Spray Test | | | | | | | | |
| EN 13697 | — | — | — | Sa > 6.27 | — | — | — | — |
| (log10 | | | | Ec > 6.25 | | | | |
| reduction) | | | | Pa > 4.97 | | | | |
| | | | | Eh > 6.56 | | | | |

| | E57 | E58 | E59 | E60 | E61 | E62 |
|---|---|---|---|---|---|---|
| CuSO₄•5H₂O | 0.1 | 0.1 | 0.1 | 0.1 | 0.1 | 0.1 |
| ethanol (100%) | 35 | 35 | 35 | 30 | 25 | 20 |
| ethanol (95%) | — | — | — | — | — | — |
| BTC-65 (50%) | 0.2 | 0.2 | 0.2 | 0.2 | — | — |
| Onyxide 3300 | — | — | — | — | 0.30 | 0.30 |
| (33%) | | | | | | |
| Neodol 91-6 | — | 0.1 | 0.1 | 0.1 | 0.1 | 0.1 |
| triethanolamine | 0.41 | 0.4 | 0.1 | 0.45 | 0.1 | 0.28 |
| ethanolamine | — | — | 0.1 | — | — | — |
| NH4OH | — | — | 0.17 | 0.21 | 0.10 | 0.18 |
| (29.8%) | | | | | | |
| NaOH (10%) | — | — | 0.25 | — | — | — |
| Citrosol | — | — | — | — | — | — |
| 502(50%) | | | | | | |
| ammonium | — | — | — | — | — | — |
| acetate | | | | | | |
| Monacor BE | — | — | — | — | 0.06 | — |
| di H₂O | 64.29 | 64.2 | 63.98 | 68.94 | 74.2 | 79.0 |
| pH | 8.1 | 8.1 | 11.03 | 9.51 | 9.21 | 9.16 |
| copper ion | 254 | 254 | 254 | 254 | 254 | 254 |
| content (ppm) | | | | | | |
| appearance | light blue | light blue | light blue | light blue | light blue | light blue |
| ASTM E 1052 | PV1 < 3.33 | PV1 ≦ 3.17 | PV1 > 5.88 | PV1 > 5.17, | PV1 ≦ 3.23 | PV1 < 3.00 |
| (log10 | | FCV > 6.0 | | HAV ≦ 2.00, | | FCV > 6.00 |
| reduction) | | | | HAdV > 4.50, | | |
| | | | | HSV-1 > 6.0 | | |
| ASTM 1053 | — | — | — | — | — | — |
| (log10 | | | | | | |
| reduction) | | | | | | |
| AOAC | — | — | — | — | — | — |
| Germicidal | | | | | | |
| Spray Test | | | | | | |
| EN 13697 | — | Sa > 6.12 | — | Sa > 6.62 | — | — |
| (log10 | | Ec > 5.66 | | Ec > 6.63 | | |
| reduction) | | Pa > 3.80 | | Pa > 6.69 | | |
| | | Eh > 6.50 | | Eh > 6.74 | | |

| | E63 | E64 | E65 | E66 |
|---|---|---|---|---|
| CuSO₄•5H₂O | 0.099 | 0.099 | 0.05 | 0.025 |
| ethanol (100%) | 45 | 45 | 45 | 45 |
| ethanol (95%) | — | — | — | — |
| BTC-65 (50%) | — | — | — | — |
| Onyxide 3300 | 0.151 | 0.076 | 0.303 | 0.303 |
| (33%) | | | | |
| Neodol 91-6 | 0.1 | 0.1 | 0.1 | 0.1 |
| triethanolamine | 0.35 | 0.28 | 0.2 | 0.1 |
| ethanolamine | — | — | — | — |
| NH4OH | 0.09 | 0.09 | 0.13 | 0.08 |
| (29.8%) | | | | |
| NaOH (10%) | — | — | — | — |
| Citrosol | — | — | — | — |
| 502(50%) | | | | |
| ammonium | — | — | — | — |
| acetate | | | | |
| Monacor BE | — | — | 0.06 | 0.06 |
| di H₂O | 54.21 | 54.35 | 54.15 | 54.33 |
| pH | 8.97 | 9.05 | 9.84 | 10.06 |
| copper ion | 254 | 254 | 127 | 64 |
| content (ppm) | | | | |
| appearance | light blue | light blue | light blue | light blue |
| ASTM E 1052 | PV1 > 5.67 | PV1 > 5.67 | PV1 > 5.67 | PV1 > 5.67 |
| (log10 | | | | |
| reduction) | | | | |
| ASTM 1053 | — | — | — | — |
| (log10 | | | | |
| reduction) | | | | |
| AOAC | — | — | — | — |
| Germicidal | | | | |
| Spray Test | | | | |
| EN 13697 | — | — | — | — |
| (log10 | | | | |
| reduction) | | | | |
| | E67 | E68 | E69 | E70 |
| CuSO₄•5H₂O | — | — | — | — |
| CuCl₂•2H₂O | 0.1 | — | — | — |
| Cu(OAcO)₂• | — | 0.1 | — | — |
| H₂O | | | | |
| Cu D-gluconate | — | — | 0.1 | — |
| Cu(I)Cl•H₂O | — | — | — | 0.1 |
| ethanol (100%) | 45 | 45 | 45 | 45 |
| ethanol (95%) | — | — | — | — |
| BTC-65 (50%) | 0.2 | 0.2 | 0.2 | — |
| Onyxide 3300 | — | — | — | 0.3 |
| (33%) | | | | |
| Neodol 91-6 | 0.1 | 0.1 | 0.1 | 0.1 |
| triethanolamine | 0.6 | 0.45 | 0.45 | 0.45 |
| ethanolamine | — | — | — | — |
| NH4OH | — | — | — | — |
| (29.8%) | | | | |
| NaOH (10%) | — | — | — | — |
| Citrosol | — | — | — | — |
| 502(50%) | | | | |
| di H₂O | 54.0 | 54.15 | 54.35 | 54.05 |
| pH | 8.1 | 8.03 | 8.03 | 8.14 |
| copper ion | 372 | 318 | 140 | 641 |
| content (ppm) | | | | |
| appearance | light blue | light blue | — | light blue, |
| | | | | slight haze |
| ASTM E 1052 | PV1 > 6.00 | PV1 > 6.00 | PV1 ≦ 5.50 | PV! > 6.00 |
| (log10 | | | | FCV => 6.00 |
| reduction) | | | | |
| ASTM 1053 | — | — | — | — |
| (log10 | | | | |
| reduction) | | | | |
| EN 13697 | — | — | — | — |
| (log10 | | | | |
| reduction) | | | | |

As can be seen from the foregoing results the compositions of the invention exhibited excellent microbicidal efficacy as demonstrated by the various test results, even wherein reduced levels of ethanol was present as a constituent.

Although this invention has been shown and described with respect to the detailed embodiments thereof, it will be understood by those of ordinary skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the appended claims.

1. A liquid, inanimate surface treatment composition which imparts a microbicidal benefit to treated surfaces which composition comprises:
a copper source material which releases copper ions into the treatment composition, preferably a source of Cu(I) and/or Cu(II) ions;
at least one lower alkyl aliphatic monohydric alcohol;
water;
optionally, one or more further constituents which impart one or more advantageous technical or aesthetic benefits to the compositions, including one or more detersive surfactants;
wherein the composition excludes quaternary ammonium surfactant compounds,
wherein the composition has a pH of at least 5,
wherein the surface treatment compositions are characterized in exhibiting a microbicidal benefit when tested against one or more challenge microorganisms according to one or more of the following standardized test protocols: ASTM E1052 Standard Test Method for Efficacy of Antimicrobial Agents against Viruses in Suspension, or ASTM E1053 Standard Test Method to Assess Virucidal Activity of Chemicals Intended for Disinfection of Inanimate, Nonporous Environmental Surfaces, or European Standard Surface Test, EN1369, or AOAC Germicidal Spray Products as Disinfectant Test Method, AOAC Index, 17^{th} Ed. (2000) preferably against poliovirus type 1 (Sabin) (“PV1”).

2. A liquid, inanimate surface treatment composition which imparts a microbicidal benefit to such treated surfaces which compositions comprises:
a copper source material which releases copper ions into the treatment composition;
in excess of 0% wt., and up to but excluding 20% wt. of at least one lower alkyl aliphatic monohydric alcohol;
water;
optionally, one or more further constituents which impart one or more advantageous technical or aesthetic benefits to the compositions, including one or more detersive surfactants;
wherein the composition excludes quaternary ammonium surfactant compounds,
wherein the composition has a pH of at least 5,
wherein the surface treatment compositions are characterized in exhibiting a microbicidal benefit when tested against one or more challenge microorganisms according to one or more of the following standardized test protocols: ASTM E1052 Standard Test Method for Efficacy of Antimicrobial Agents against Viruses in Suspension, or ASTM E1053 Standard Test Method to Assess Virucidal Activity of Chemicals Intended for Disinfection of Inanimate, Nonporous Environmental Surfaces, or European Standard Surface Test, EN1369, or AOAC Germicidal Spray Products as Disinfectant Test Method, AOAC Index, 17^{th} Ed. (2000) against one or more challenge microorganisms, especially preferably against poliovirus type 1 (Sabin) (“PV1”).

3. A liquid, inanimate surface treatment composition which imparts a microbicidal benefit to such treated surfaces which compositions comprises:
a copper source material which releases copper ions into the treatment composition, preferably a source of Cu(I) and/or Cu(II) ions;
at least one lower alkyl aliphatic monohydric alcohol;
at least one further detersive surfactant, other than a quaternary ammonium surfactant compound which provides a microbicidal benefit;
water;
optionally, one or more further constituents which impart one or more advantageous technical or aesthetic benefits to the compositions, including one or more detersive surfactants;
wherein the composition excludes quaternary ammonium surfactant compounds,
wherein the composition has a pH of at least 5,
wherein the surface treatment compositions are characterized in exhibiting a microbicidal benefit when tested according to one or more of the following standardized test protocols: ASTM E1052 - 96(2002) Standard Test Method for Efficacy of Antimicrobial Agents against Viruses in Suspension, or ASTM E1053-11 Standard Test Method to Assess Virucidal Activity of Chemicals Intended for Disinfection of Inanimate, Nonporous Environmental Surfaces, or European Standard Surface Test, EN1369, or AOAC Germicidal Spray Products as Disinfectant Test Method, AOAC Index, 17^{th} Ed. (2000) against one or more challenge microorganisms, especially preferably against Poliovirus type 1 (Sabin) (“PV1”).

4. A liquid, inanimate surface treatment composition which imparts a microbicidal benefit to such treated surfaces which compositions comprises (or in certain preferred embodiments may consist essentially of, or may consist of):
a copper source material which releases copper ions into the treatment composition, preferably a source of Cu(I) and/or Cu(II) ions;
at least one lower alkyl aliphatic monohydric alcohol;
at least one further detersive surfactant, other than a quaternary ammonium surfactant compound which provides a microbicidal benefit, which further detersive surfactant is preferably at least one nonionic surfactant;
water;
optionally, one or more further constituents which impart one or more advantageous technical or aesthetic benefits to the compositions, including one or more detersive surfactants;
wherein the composition excludes quaternary ammonium surfactant compounds,
wherein the composition has a pH of at least 5,
wherein the surface treatment compositions are characterized in exhibiting a microbicidal benefit when tested according to one or more of the following standardized test protocols: ASTM E1052 Standard Test Method for Efficacy of Antimicrobial Agents against Viruses in Suspension, or ASTM E1053 Standard Test Method to Assess Virucidal Activity of Chemicals Intended for Disinfection of Inanimate, Nonporous Environmental Surfaces, or European Standard Surface Test, EN1369, or AOAC Germicidal Spray Products as Disinfectant Test Method, AOAC Index, 17′¹¹ Ed. (2000) against one or more challenge microorganisms, especially preferably against Poliovirus type 1 (Sabin) (“PV1”).

5. A composition according to claim 1, wherein the composition exhibits a pH of at least at least 6.

6. A composition according to claim 1, wherein ethanol is the predominant alcohol present in the lower alkyl aliphatic monohydric alcohol constituent.

7. A composition according to claim 6 wherein ethanol is the sole alcohol present in the lower aliphatic monohydric alcohol constituent.

8. A microbicidal control system of constituents which comprises: water, one or more one or more Cᵢ-C₄ aliphatic alcohols in an amount of up to, but less than 20% wt., and especially preferably wherein ethanol is the predominant or sole C₁-C₄ aliphatic alcohols present, and, where necessary, a buffer or pH adjusting agent to impart an alkaline pH, preferably an alkaline pH of 7.5 or greater, and optionally one or more further constituents which may or may not provide a further microbicidal benefit, and further wherein the composition excludes quaternary ammonium surfactant compounds,

9. A microbial control system according to claim 8, wherein the microbial control system further includes at least one further detersive surfactant, other than a quaternary ammonium surfactant compound which provides a microbicidal benefit, which further detersive surfactant is preferably at least one nonionic surfactant.

10. A method of controlling the incidence of undesired microorganisms on an inanimate surface, the method comprising the step of:
contacting the inanimate surface which is in need of treatment or upon which the presence of one or more undesirable microorganisms are suspected or are known to be present, with an effective amount of a liquid, inanimate surface treatment composition according to claim 1, to provide a microbicidal benefit to the contacted surface.

11. A method of controlling the incidence of undesired microorganisms in air, or in a headspace such as the ambient air within a closed volume such as a room or the interior of a vehicle, the method comprising the step of: delivering and dispersing within an airspace an effective amount of a liquid, inanimate surface treatment composition according to claim 1 to provide a microbicidal benefit to the treated air, preferably to provide a microbicidal benefit to the treated air.

12. A composition according to claim 5, wherein the composition exhibits a pH of at least at least 7.

13. A composition according to claim 2, wherein the composition exhibits a pH of at least at least 6.

14. A composition according to claim 13, wherein the composition exhibits a pH of at least at least 7.

15. A composition according to claim 3, wherein the composition exhibits a pH of at least at least 6.

16. A composition according to claim 15, wherein the composition exhibits a pH of at least at least 7.

17. A composition according to claim 4, wherein the composition exhibits a pH of at least at least 6.

18. A composition according to claim 17, wherein the composition exhibits a pH at least at least 7.
